(19) United States
(12) Patent Application Publication
(10) Pub. No.: US 2022/0319656 A1
(43) Pub. Date: Oct. 6, 2022

(54) AUTOMATED LITERATURE META ANALYSIS USING HYPOTHESIS GENERATORS AND AUTOMATED SEARCH

(71) Applicant: TECHNION RESEARCH & DEVELOPMENT FOUNDATION, Haifa (IL)

(72) Inventors: Yosef Shamay, Zichron Yaakov (IL); David Dobreen, Kfar Saba (IL)

(73) Assignee: TECHNION RESEARCH & DEVELOPMENT FOUNDATION, Haifa (IL)

(21) Appl. No.: 17/633,701

(22) PCT Filed: Aug. 16, 2020
(86) PCT No.: PCT/IL2020/050899

§ 371 Date: Feb. 8, 2022

Related U.S. Application Data
(60) Provisional application No. 62/889,115, filed on Aug. 20, 2019.

(51) Int. Cl. G16H 20/00; G16H 70/20; G06N 5/04

(52) U.S. Cl. CPC G16H 20/00; G16H 70/20; G06N 5/041

(57) ABSTRACT

Provided herein are methods and systems for automated generation of hypothesis based on sets of search terms, and scoring of said automatically generated hypothesis to determine novelty, reasonability and/or feasibility thereof. Further provided are methods of utilizing said generated hypothesis for determination of personalized treatment regime of various health conditions.

### TECHNICAL FIELD

The present disclosure relates generally to systems and methods for automatic meta-analysis of data for generating and scoring hypotheses.

### BACKGROUND

An enormous amount of scientific and clinical data is generated, by scientists, for example, in the form of manuscripts, papers, books, clinical trial reports and patents, which is stored in large database and most commonly accessed using search engines or data bases, such as PubMed or Google Scholar.

Technological developments in text and data mining (TDM) have opened up a wealth of new possibilities for researchers, enabling the analysis of textual information in ways that were not previously feasible. TDM can be used to extract and display information in a structured, machine-readable way that makes it easier to process and compare with other sources of data. In the biomedical field, automated literature search and TDM is used to identify relationship and interactions between diseases, genes, proteins and drugs and can save time and effort both scientists and clinicians. Most TDM methods rely on natural language processing where the effort of computation is focused on reading, deciphering and understanding human languages in the scientific text a valuable manner. The current solutions for automated literature review are mainly focused on summarizing big textual data and presenting conclusions with as little as possible information so it can be humanly perceived. Several of these tools use unique visual output of literature search to facilitate perception of the scientific landscape related to the search. For examples, CoreMine-Medical, Science.gov, Embase, SciFinder, and the like, are aimed to deliver small and valuable information from multiple scientific papers in a visual way such as connection between concepts in papers and intensity of connection according to the strength of connection. Even though these tools enhance scientific literature search, and can speed up the process by providing more relevant searches they cannot present a full detailed picture of what is known and more importantly what is unknown in a scientific field or in relation to a scientific problem.

With all the wealth of available information, it has become practically impossible for individuals to perceive what is known in a scientific field using conventional literature review methods. It is even more difficult for scientists to perceive what is still unknown in a scientific field and which scientific hypotheses have not been tested and published yet. Furthermore, even though there are various tools to search and summarize data in scientific databases using TDM approaches, there is no reliable method that can present users a map of the known hypotheses space together with the unknown, for the purpose of facilitating scientific discoveries.

Thus, there is a need in the art for automated tools that can generate and present a map of known hypotheses space along with the unknown, and which can further allow ranking the generated hypotheses to increase the assessment thereof.

### SUMMARY

Aspects of the disclosure, according to some embodiments thereof, relate to advantageous systems and method for automated literature meta-analysis (also referred to herein as “ALMA”) for the generation of hypotheses, which can further be ranked or scored based on various parameters, such as, novelty, reasonability and/or feasibility.

In some embodiments, the systems and methods disclosed herein are advantageous as they can allow a user to identify hypotheses in various scientific fields using sets of search terms selected by a used, wherein the generated hypotheses may otherwise would not have been suggested or recognized. Furthermore, the systems and methods disclosed herein can advantageously allow the ranking of the generated hypotheses to provide further input regarding their novelty, feasibility and/or reasonability. The disclosed systems are both cost and time effective.

According to some embodiments, without wishing to be bound by any theory, the disclosed systems and methods are based on the frequency of co-occurrence of search terms (words/strings) in scientific literature. In some embodiments, when two search term (for example, words) appear together many times they can be considered to ‘go together’ or be associated. In some embodiments, this association premise may be expanded into the following: a true scientific hypothesis occurs more than a false scientific hypothesis in the literature, and/or is persistent in time. Statistically wise, a true hypothesis would have a higher number of publications then false hypothesis or an unknown hypothesis. Since hypotheses, as used herein, are a combination of search terms (such as words), the disclosed hypothesis generator is utilized and coupled to an automated search in order to visualize the frequency of published hypotheses next to unpublished. In some embodiments, analyzing the temporal frequency of published hypotheses can indicate false or true classification.

In some embodiments, the systems and methods disclosed herein can further be used to generate not merely scientific hypotheses, but to further generate suggested detailed treatment plans, such as high resolution combination therapy (HRCT). The treatment plans that may be generated as disclosed herein, are advantageous, as they can be personalized to specific patients, based on the specific parameters of the patient. Thus, the systems and methods disclosed herein can be used to automatically generate personalized treatment plans, based on the specific characteristic of the patient, and the respective scientific knowledge. In some embodiments, the provided methods can advantageously automatically integrate hundreds of scientific findings into a personalized, complex and highly detailed treatment plan while ranking the elements of the plan by novelty/risk, reasonability and feasibility.

According to some embodiments, the systems and methods disclosed herein are advantageous over currently used text and data mining (TDM) methods, which are based on natural language processing (NLP). These methods aim to ‘teach’ the computerized system how to read scientific papers using sophisticated statistical training of human annotations. In contrast, the currently disclosed methods and systems are for automated literature meta-analysis (ALMA).

According to some embodiments, the methods disclosed herein include computerized search tools which include a hypothesis generator, generating multiple hypotheses in more than one step. In order to evaluate the known and known spaces from three types of databases/search sets (for example gene, disease, drug), two-steps of hypotheses generation may be required. In some embodiments, a first hypothesis stage may evaluate the relations (for example, by citation (or the NOP) rating score) between, for example, gene and disease, and a second hypothesis stage may evaluate the relations of each disease-gene combination and a drug. Additional hypotheses can further evaluate, for example, the combination gene, disease, drug with, for example, terms such as, encapsulation ingredient, clinical trials, radiotherapy, immunotherapy and other related variables.

According to further embodiments, the method disclosed herein can advantageously further allow multiple hypotheses evaluations, based on number of “hits” or “citations” resulting from the automatic search t to identify knowledge spaces of known versus unknown but having high probability to be true, based on the published knowledge, as detailed herein below.

According to further embodiments, the systems and methods disclosed herein are advantageous as it can allow perceiving and presenting, based on a minimal prior preparation, the known scientific space, together with the unknown. The disclosed systems and methods can easily identify and present hypotheses and combinations that are of high value based on their prevalent appearance in the global knowledge and those that are most probably of high value although they are not yet part the global knowledge.

According to some embodiments, the methods disclosed herein are not used merely for entirely literature review but to point out which hypothesis can/should be followed up. Using manual searches it would be very hard to do a comprehensive literature search and see all that is known and unknown and more importantly visualizing it, to facilitate targeted literature search and promote discoveries.

According to some embodiments, the disclosed methods can be used to visually display the knowns and unknowns in scientific literature, to thereby facilitate the identification of new scientific hypothesis. In some embodiments, the methods can advantageously be used to can rank the hypotheses by reasonability, feasibility, complexity, and/or novelty.

Thus, according to some embodiments, there is provided a method for generation and ranking of hypotheses, based on one or more sets of search terms, the method includes one or more of the steps of:
obtaining one or more sets of two or more search terms (including, for example, words, sentences, phrases, and the like);
generating multiple hypotheses, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters; and
ranking the selected generated hypotheses based on the NOP matrix, wherein the ranking is indicative of the degree of novelty and/or degree of feasibility and/or degree of reasonability of the selected generated hypothesis.

According to some embodiments, there is provided a method for generation and ranking of various hypotheses, based on a set of search terms determined by a user, wherein the method may include one or more of the steps of:
obtaining two or more sets of search terms (such as words, sentences, phrases, etc.);
generating combinations of search terms from the sets, wherein each combination corresponds to a potential hypothesis;
searching on one or more suitable electronic databases for each combination of search terms, to obtain the number of publications (NOP) that corresponds to the respective hypothesis;
generating a matrix (such as in the form of a table), with components/cells indexed according to the hypotheses, wherein each component is assigned a value that may equal to the NOP of the combination of search terms corresponding to the respective hypothesis;
sorting the matrix according to one or more selected sorting criteria; and
ranking at least some of the hypotheses based on the sorted matrix, wherein the ranking is indicative of the degree of novelty and/or degree of feasibility and/or degree of reasonability of the hypotheses.

According to some embodiments, the method is computer implemented.

According to some embodiments, there is provided a system which includes a processor configured to execute the method for generation and optional ranking of hypotheses, as disclosed herein. In some embodiments, the system may further include a user interface, a display unit, a communication unit, and the like. In some embodiments, the system includes a computer having one or more processors.

According to some embodiments, there is provided a computer program which includes instructions to execute the steps of the method for generation of hypotheses using automated literature meta-analysis, as disclosed herein.

According to some embodiments, there is provided a computer-readable medium having stored thereon the computer program which includes instructions to execute the steps of the method for generation of hypotheses using automated literature meta-analysis, as disclosed herein.

According to some embodiments, there is provided a method for predicting reasonability of unpublished biomedical hypotheses with automated literature meta-analysis (ALMA) to generate High Resolution Combination Therapy.

According to some embodiments, there is provided a method for automated literature meta-analysis (ALMA) for generating high resolution combination therapy.

According to some embodiments, there is provided a computer implemented method for generation and ranking of hypotheses, based on a set of search terms, the method includes one or more of the steps of:
obtaining two or more sets of search terms;
generating combinations of search terms from the sets, each combination corresponding to a hypothesis;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of the degree of novelty and/or degree of feasibility and/or degree of reasonability of the hypotheses.

According to some embodiments, the method may further include a step of performing an additional search using a second set of search terms or search variables on the sorted NOP matrix of the one or more selected generated hypotheses, to thereby generate a comparison matrix between the sorted NOP matrix and the results of the additional search.

According to some embodiments, the method may further include a step of presenting one or more of: the matrix of the NOP, the sorted matrix of the NOP, the ranking of the selected generated hypotheses, or any combination thereof.

According to some embodiments, each of the search terms may be selected from: a word, list of words, a sentence, a generic term, a question, or any combination thereof. Each possibility is a separate embodiment.

According to some embodiments, the selected combination of the search may be structured as “one vs. many”, “many vs. many”, or both.

According to some embodiments, the search may be performed using a suitable web crawler, web scraper, automated search tool, or any combination thereof. According to some embodiments, the database may be selected from PubMed, Google Scholar, clinicaltrials.gov, Embase and/or Semantic Scholars.

According to some embodiments, the NOP matrix may be visualized using a visual coding having adjustable threshold, based on the visualization parameters.

According to some embodiments, the reasonability may include local reasonability (LR), horizontal reasonability (HR), vertical reasonability (VR), or any combination thereof. In some embodiments, the reasonability may further include extended horizontal reasonability (THR) and/or extended vertical reasonability (TVR).

According to some embodiments, the reasonability may include local reasonability (LR), horizontal reasonability (HR), vertical reasonability (VR), extended horizontal reasonability (THR), extended vertical reasonability (TVR) or any combination thereof. Each possibility is a separate embodiment.

According to some embodiments the degree of feasibility and/or degree of reasonability may be determined based on an adjustable threshold of number of publications. According to some embodiments, the adjustable threshold is user defined.

According to some embodiments, the method may further include providing a numerical score based on the ranking of the hypothesis.

According to some embodiments, there is provided a computer implemented method for generation and ranking of hypotheses, based on a set of search terms, the method included one or more of the steps of:

a. obtaining a set of two or more search terms;

b. generating multiple hypotheses, based on a selected combination of the search terms;

c. performing a search for the generated hypotheses on one or more suitable databases stored on a server, to determine the number of publications (NOP) for each generated hypothesis;

d. generating a matrix of the NOP of one or more selected generated hypotheses;

e. sorting the NOP matrix of the one or more selected generated hypotheses, based on one or more sorting parameters; and

f. ranking the selected generated hypotheses based on the NOP matrix, wherein the ranking is indicative of the degree of novelty and/or degree of feasibility and/or degree of reasonability of the selected generated hypothesis.

According to some embodiments, there is provided a system for automated generation of a hypothesis, based on sets of search terms, the system includes a processor configured to execute a method which includes one or more of the steps of:
obtaining two or more sets of search terms;
generating combinations of search terms from the sets, each combination corresponding to a hypothesis;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of the degree of novelty and/or degree of feasibility and/or degree of reasonability of the hypotheses.

According to some embodiments, there is provided a system for automated generation of a hypothesis, based on sets of search terms, the system includes a processor configured to execute a method which includes one or more of the steps of:
obtaining a set of two or more search terms;
generating multiple hypotheses, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters; and
ranking the selected generated hypotheses based on the NOP matrix, wherein the ranking is indicative of the degree of novelty and/or degree of feasibility and/or degree of reasonability of the selected generated hypothesis.

According to some embodiments, the systems disclosed herein may further include one or more of: a user interface unit, a display unit, a communication unit, or any combination thereof.

According to some embodiments, there is provided a computer-readable medium having stored thereon instructions to execute the steps of a method for generation and ranking of hypotheses, based on a set of search terms, the method includes one or more of the steps of:
obtaining two or more sets of search terms;
generating combinations of search terms from the sets, each combination corresponding to a hypothesis;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of the degree of novelty and/or degree of feasibility and/or degree of reasonability of the hypotheses.

According to some embodiments, there is provided a computer-readable medium having stored thereon instructions to execute the steps of a method for generation and ranking of hypotheses, based on a set of search terms, the method included one or more of the steps of:
obtaining a set of two or more search terms;
generating multiple hypotheses, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters; and
ranking the selected generated hypotheses based on the NOP matrix, wherein the ranking is indicative of the degree of novelty and/or degree of feasibility and/or degree of reasonability of the selected generated hypothesis.

A computer implemented method for determining a personalized high resolution treatment regime of a patient afflicted with a disease, the method comprising:
obtaining a set of two or more search terms related to the disease of the patient;
generating multiple hypotheses related to treatment of the disease, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters;
ranking the selected generated hypotheses based on the NOP matrix, wherein the ranking is indicative of the degree of novelty and/or degree of feasibility and/or degree of reasonability of the selected generated hypothesis, to determine a first treatment;
repeating the search for one or more times with search terms related to the disease and/or the first treatment, to determine an additional one or more treatments; and
determining, based on the identified treatments, a personalized treatment regime for said patient.

According to some embodiments, there is provided a computer implemented method for determining a personalized high resolution treatment regime of a patient afflicted with a disease, the method includes one or more of the steps of:
obtaining two or more sets of search terms;
generating combinations of search terms from the sets, each combination corresponding to a hypothesis related to treatment of the disease;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of the degree of novelty and/or degree of feasibility and/or degree of reasonability of the hypotheses, to determine a first treatment;
repeating the search for one or more times with search terms related to the disease and/or the first treatment, to determine an additional one or more treatments; and
determining, based on the identified treatments, a personalized treatment regime for said patient.

According to some embodiments, the determined treatment is a combination therapy. In some embodiments, the patient is a cancer patient.

According to some embodiments, the first treatment and/or the one or more additional treatments may be selected from: a drug, an immunotherapy, a surgical procedure, radiotherapy, chemotherapy, psychotherapy, lifestyle therapy, or any combination thereof. Each possibility is a separate embodiment.

According to some embodiments, the treatment regime may further include a spatial distribution sequence of the first and/or additional treatment.

According to some embodiments, there is provided a system for determining a personalized high resolution treatment regime of a patient afflicted with a disease, the system includes a processor configured to execute the steps of the method for determining a personalized high resolution treatment regime of a patient afflicted with a disease.

According to some embodiments, there is provided a computer-readable medium having stored thereon instructions to execute the steps of a method for determining a personalized high resolution treatment regime of a patient afflicted with a disease.

According to some embodiments, there are provided methods and systems for visualization of temporal landscape and/or geographical distribution of hypotheses.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1 illustrates steps in a method for automated literature meta-analysis, according to some embodiments;

FIGS. 2A-B illustrate exemplary steps 1-3 in a method for automated literature meta-analysis (ALMA) and exemplary implantation thereof, according to some embodiments.

FIG. 2A—shows a schematic representation of steps 1-3 in ALMA. FIG. 2B shows an example for an automatic search of all 1800 FDA approved drugs together with a rare disease (uveal melanoma).

FIG. 3 illustrates an example of the results of automated literature meta analysis (ALMA) in a form of a matrix, according to some embodiments. The search is comprised of sets of various search terms (cancers and drug treatments with the focus of the proto-oncogene BRAF). In the results presented in the enlarged, right hand table depicted in FIG. 3, the terms Vemurafenib, cobimetinib, clinical trial, nivolumab (single search) were excluded from the matrix to simplify the presentation.

FIGS. 4A-D illustrate examples of “One vs Many” structured searches, using automated literature meta analysis (ALMA), according to some embodiments. FIG. 4A—Generating a list of common genes in uveal melanoma disease, using ALMA; FIG. 4B—Comparison of Uveal melanoma disease and renal cell carcinoma (RCC) disease. FIG. 4C—a graph showing an overlay of uveal melanoma results on RCC results. The genes presented are sorted by the normalized Number of Publications (NOP) value in uveal melanoma. FIG. 4D—Further examples of “One vs. Many” questions, which can be searched and answered using the automated literature meta analysis. KI=Kinase inhibitor, EPFL=Ecole polytechnique fédérale de Lausanne.

FIGS. 5A-D illustrate examples of “Many vs Many” structured searches, using automated literature meta analysis (ALMA), according to some embodiments. FIG. 5A-Sorting of 11,000 potential drug cancer combinations of hypotheses, based on the sum of the cells in columns. The text in the enlarged text boxes details the hypothesis in the respective boxed cell. FIG. 5B—sorting the hypothesis matrix with clustering by weighing based on rows or columns, as indicated. The text in the enlarged text boxes details the hypothesis in the respective boxed cell. FIG. 5C Automated search of 400 cancer genes with 16 cancer. Vertical normalization and sorting by cancer shows the most studied gene per cancer. FIG. 5D—Focused representation of the normalized matrix with 12 cancers and 12 genes. NOP=number of publications.

FIGS. 6A-B illustrate examples of cancer nanomedicine structured searches, using automated literature meta analysis (ALMA), according to some embodiments. FIG. 6A-Preparation of a Hypotheses matrix structured as: cancer types/drugs/and the variable search term (word) “nanoparticle”. The obtained merged matrix presented in FIG. 6A contains the NOPs of all the cancer-drug combinations, with and without the variable (var) “nanoparticle” side by side. FIG. 6B shows Enlarged section of the matrix with the strongest cancers/drugs hypotheses. Dark shade (originally Red) indicates 0 publications and dark gray shades (originally dark green) indicates more than 20 publications. Dark cells (originally presented as Red cells) next to dark gray cells (originally presented as dark green cells) are indicative of a hypothesis that is novel (i.e., never been published) but should be potentially reasonable. If there are gray (originally green) and var cells in the row of that hypothesis then it is indicative that the hypothesis is also feasible.

FIGS. 7A-B illustrates examples of personalized cancer nanomedicine structured searches, using automated literature meta analysis (ALMA), according to some embodiments. FIG. 7A—shows a sorted hypotheses matrix generated (structured) using search terms: genes/drugs/and a cancer type, followed by the variable search term “nanoparticle”. The merged matrix contains the NOPs of all the cancer-drug combinations with and without the variable (var) “nanoparticle” side by side. FIG. 7B-Enlarged section with the strongest cancers/drugs hypotheses. Numbers are NOPs of hypotheses. Dark cells (originally Red) indicates 0 publications and dark gray cells (originally dark green) indicates more than 20 publications. Dark cells (originally presented as Red cells) next to dark gray cells (originally presented as dark green cells) are indicative of a hypothesis that is novel (i.e., never been published) but should be potentially reasonable. If there are gray (originally green) and var cells in the row of that hypothesis then it is indicative that the hypothesis is also feasible.

FIG. 8 shows example of defining hypothesis descriptors of novelty and reasonability in a merged comparison matrix, generated using automated literature meta analysis (ALMA), according to some embodiments. The example shows the use of various descriptors to rank hypotheses (for example by novelty (N), LR=Local Reasonability (LR), HR (Horizontal Reasonability) and/or vertical Reasonability (VR)), which can be indicative of the characteristics (such as, strength) of a selective hypothesis.

FIGS. 9A-C show examples of evaluating the score of novelty and reasonability of hypothesis descriptors of novelty and reasonability in a merged comparison matrix, generated using automated literature meta analysis (ALMA), according to some embodiments. FIG. 9A—shows a generated merged comparison matrix. FIG. 9B—for each cell in the matrix (table) the descriptors of Novelty (N), Local Reasonability (LR), Horizontal Reasonability (HR) and/or Vertical Reasonability (VR) are calculated, using predetermined thresholds applied by the user (similarly to the colorization of matrix as detailed above, while using High and medium thresholds)) and presented in the Table shown in FIG. 9B. FIG. 9C—The hypotheses (cells in the matrix/table) are ranked, based on user-defined priorities. In the table shown in FIG. 9C, the hypotheses are ranked by N followed by VR, HR and LR, to identify the most novel, most reasonable and feasible hypotheses.

FIGS. 10A-D show examples of finding novel and reasonable hypotheses with comparison matrix and triangulation, according to some embodiments. FIG. 10A shows the Number of publications (NOP) of 23 kinase inhibitors (KIs), combined with head and neck squamous cell carcinoma (HNSCC). FIG. 10B shows that the addition of concepts, ‘radiotherapy’ and ‘nanoparticle’ generates a comparison matrix of all 3 elements (KI, HNSCC, Radiotherapy). NOP of every possible combination: Lighter gray (originally green) is KI-Radiotherapy (horizontal reasonability), light gray (originally orange) is KI-HNSCC (local reasonability), darker gray (originally blue) is HNSCC-Radiotherapy (vertical reasonability) and dark gray (originally red) is the combined KI-HNSCC-Radiotherapy (novelty candidate). The same procedure was repeated with the string ‘nanoparticle’. FIG. 10C shows the ranking of hypotheses according to their novelty score (<1 publications) and reasonability score (>10 publications in every dual combination). FIG. 10D illustrate the Triangulation method used to identify novel and reasonable hypotheses in 7 cancers and 50 kinases, ranked by the highest score of novelty and reasonability.

FIG. 11A—illustrates a scheme of a method for identifying novel experiments based on inventory of available drugs and cell lines (e.g., those that are available in the lab) and various variables, utilizing automated literature meta analysis (ALMA);

FIG. 11B—a scheme showing generation of a comparison matrix of 50 drugs and 15 cell lines (available in the lab) with additional variable search terms (words), including ‘osteosarcoma’ and ‘nanoparticle’. The top 12 drugs and 2 cell lines were selected for further search;

FIG. 11C—shows comparison tables of the NOP matrix to cell viability experiments with matching drugs in MG63 and Fadu cells. The cells were incubated with the indicated drugs for 72 hours and viability was measured with MTT assay;

FIG. 11D shows representative DLS size measurement graphs of Car-INP. Further shown are pictograms of free Car and Car-INP in water in Eppendorf test tubes;

FIG. 11E shows a line graph of the Car-INP surface zeta potential distribution;

FIG. 11F shows line graphs of MTT assay results of cell viability of MG63 and Fadu cells incubated with Carfilzomib and Car-INP for 72 h.

FIG. 11G shows representative fluorescence microscopy images uptake of Car-INP in Fadu or MG63 cells. Nanoparticles (originally shown in red) were incubated for 2 hours and stained with Hoechst for nuclear staining (originally blue);

FIG. 11H shows Brightfield images of MG63 cells with Car-INP at t=0 and 72 hours (72 h) after incubation. The experiments presented in FIGS. 11C-11H were performed in triplicates. Scale bar=25 μm. Graphs are of mean±SD

FIGS. 12A-G—Finding novel and reasonable hypotheses of molecular targeted biomaterial for multiple diseases. FIG. 12A shows a scheme of a method for identifying novel and reasonable hypotheses involving a molecularly targeted biomaterial for a certain disease, utilizing ALMA. FIG. 12B shows a search matrix table of 9 diseases with 4 types of biomaterials, used as a basis for multiple comparison matrices with the listed molecular targets (bottom right). FIG. 12C shows the ranking table of hypotheses according to their novelty score (i.e. <1 publications) and reasonability score (i.e. >10 publications in every pair combination). FIG. 12D shows pictograms of immunohistochemistry staining of ANXA1 in healthy and pancreatic patients using two different ANXA1 antibodies to provide experimental validation of reasonability for the first hypothesis presented in FIG. 12C. FIG. 12E shows pictograms of U2OS cells stained with two ANXA1 antibodies, to identify the cellular expression of ANXA1 in the cells. FIG. 12F shows bar graphs of comparison of expression of ANXA1 in different cancer patients. FIG. 12G shows survival probability (Kaplan-Mayer curves) of patients with high and low expression of ANXA1. The Data used in FIGS. 12D-12G was obtained from Human Protein Atlas database.

FIGS. 13A-C show graphs demonstrating yearly publication numbers of different cancers together with different search terms (variables). FIG. 13A shows variables of traditional pillars of cancer treatments (chemotherapy and radiotherapy). FIG. 13B shows emerging concept of novel treatments that are based on immunotherapy using the targets: PD-1 and CTLA-4; FIG. 13C shows mixed trends that are specific for the tumor types.

FIGS. 14A-D—Temporal and geographical analysis of cancer related hypotheses. FIG. 14A shows a search matrix which was generated as follows: 333 drug cancer hypotheses combinations that were generated with ALMA (based on 37 drugs and 9 types of cancer as the text search words). The obtained combinations were then used to generate the search matrix with past 6 years of publication date for the generated hypotheses. The matrix was normalized per hypothesis (horizontally) and then sorted by year 2019. FIG. 14B shows bar graphs of focused representation of three main types of temporal trends: trending up (left hand graph), stable (middle graph) and decline (right hand graph). FIG. 14C shows temporal NOP plots (number of publications per year (publication date), of one representative hypothesis of each of the graphs presented in FIG. 14B. FIG. 14D shows a matrix which includes the geographic distribution of 140 cancer ‘type-treatment type’ combination in 19 countries, normalized per hypothesis and sorted by countries (top panel). Focused representation of 15 pairs in 7 countries showing the variety of country sorted hypotheses is presented in the lower panel of FIG. 14D.

FIG. 15 shows an exemplary sorted matrix generated utilizing ALMA, of drugs having novelty and high reasonability to be active against COVID-19 infection, based on the NOP of their effect in COVID-19 related conditions.

FIG. 16 shows a schematic framework for determining an exemplary proposed High Resolution Combination Therapy (HRCT), generated based on an automated literature meta analysis (ALMA), according to some embodiments. By utilizing the appropriate sets of search terms, with ALMA, a treatment protocol, which optimizes every element in the treatment plan in a recursive manner can be generated. The treatment plan may be personalized to a specific patient.

FIGS. 17A-B show schematic illustrations of treatment plan (sequence), generated using automated literature meta analysis (ALMA), according to some embodiments. In FIG. 17A, lead treatment sequences that were identified using ALMA are presented. FIG. 17B shows cartoon illustration of an exemplary antiangiogenic treatment sequence, which normalize vessels and blood flow which helps chemotherapy to reduce tumor mass, then radiotherapy cause an inflammation in the tumor which helps immunotherapy to induce T-cell infiltration.

FIG. 18 is a schematic illustration of an output example of a HRCT protocol/plan for a lung cancer patient, the protocol generated using automated literature meta analysis (ALMA), according to some embodiments. As shown in FIG. 18, the lung cancer patient is a stage 2 cancer patient, having a KRAS and PTEN mutated genes. The detailed protocol plan includes, inter alia, dietary recommendations, activity recommendation, specific treatment regime, including type of treatment, duration and temporal distribution thereof.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, there are provided systems and methods for the generation of hypotheses using automated literature meta-analysis. In some embodiments, as further exemplified herein, the systems and methods may further be used to rank the hypothesis, based on various selected parameters, such as, for example, novelty, reasonability and/or feasibility.

According to some embodiments, the method may thus include one or more of the steps of:

1) Generating Multiple hypothesis using a hypothesis generator according to subject of interest (gene, disease, drug, treatment, plants, chemicals, formulation methods);

2) Automated literature search for ‘true’ hypotheses using a unique web crawler/scraper that extract the number of papers/results per hypothesis;

3) Analyzing, sorting and ranking of hypotheses/statements—initial presentation of known (true) hypothesis;

4) Generation of new hypotheses with the addition of text variables to top ranking hypothesis and generating multiple new hypothesis. Steps 2-4 may be repeated for a multiplicity of time. Additionally, or alternatively, this can also be done by combining results of two parallel searches into a third search.

5) Final analysis—the results are automatically sorted and ranked by the strongest hypothesis with the initial subject of interest and present a map in a form of matrix (a review matrix) containing all of the quantitative results from the multiple hypothesis searched. Color-coding may be used to facilitate user perception/review of the information. In some embodiments, hypotheses that are closer to the strongest hypothesis are potentially true even if they have no publications (i.e. zero NOP).

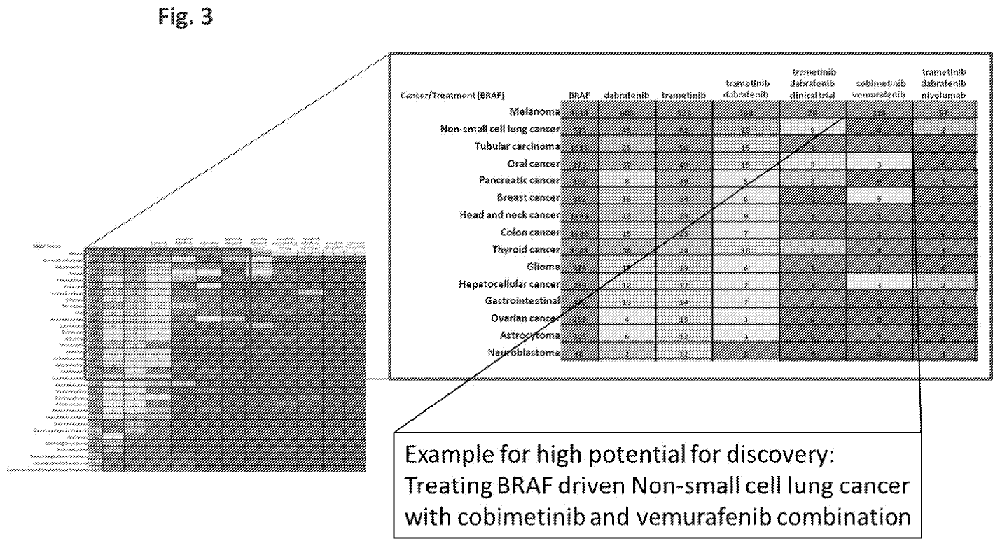

According to some embodiments, the methods disclosed herein include at least two major components: automated literature search of multiple hypotheses that were generated automatically, and an automated analysis of the results based on the concept that after sorting of the review matrix, the distance to the strongest hypothesis indicates scientific potential and feasibility. This is exemplified herein in Example 2 (FIGS. 3A-B).

In some embodiments, the methods and systems disclosed herein may be based on a principle/assumption/premise that in the scientific literature, true statements or hypotheses appear more (quantitatively) than false statements. For example, comparing the number of search results of the search set format “Drug X is used in Disease Y” using search terms “Gemcitabine is used in Pancreatic Cancer” (5886 publications in PubMed) vs “Alfacalcidol is used in Pancreatic cancer” (0 publications in Pubmed), indicates that indeed, gemcitabine which is a gold standard in pancreatic cancer treatment (and Alfacalcidol is used in Osteoporosis (585 results).

According to some embodiments, the methods are computer implemented and can generate hypotheses based on combination of sets of at least two search terms. In some embodiments, the generated hypotheses are presented in the form of a matrix, that can be sorted at will by a user, based on any selected parameter. In some embodiments, the systems and methods disclosed herein can further be used to rank the generated hypotheses, to advantageously provide a user further valuable information regarding the generated hypotheses, that otherwise would not have been available to the user.

According to some embodiments, the matrix may have any number of dimensions, including, for example, one dimension, two dimensions, three dimensions, etc., depending on the search terms, search sets and the relations there between. In some embodiments, the matrix may be in the form of a table. In some embodiments, the matrix may be in the form of a list. In some embodiments, the matrix may be in the form of a structured array. In some embodiments, the matrix may be sorted based on any desired parameter or descriptor. In some embodiments, the matrix may be sorted based on one or more parameters descriptors, including but not limited to: number of publications (NOP), Novelty (N), Local Reasonability (LR), Horizontal Reasonability (HR), Vertical Reasonability (VR), Extended Horizontal Reasonability (HR), Extended Vertical Reasonability (VR), and the like, or any combination thereof. Each possibility is a separate embodiment. In some embodiments, the matrix may be sorted by triangulation.

According to some embodiments, the matrix may be presented to a user in any appropriate means, including, in the form of text, numbers, tables, graphs, etc. In some embodiments, the matrix may be presented using color coding.

In some embodiments, the matrix may be sorted based on a threshold. In some embodiments, the threshold may be predetermined value, per each search and/or per each sub search. In some embodiments, the threshold may be user defined, per each search and/or per each sub search. In some embodiments, the threshold may be a sensitivity threshold, which may be based on input from the user, to allow, for example, for optimal clustering, according to the user.

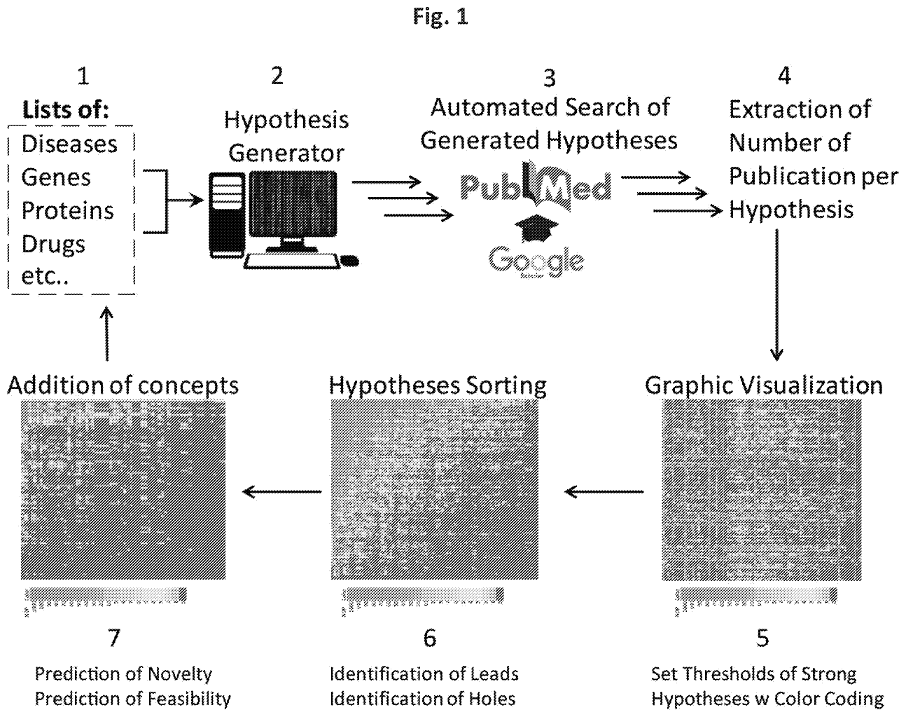

Reference is now made to FIG. 1 which schematically depicts steps in a method automated literature meta-analysis for generation of hypotheses, according to some embodiments. As shown in FIG. 1, in the first step (1)—sets of search terms (at least two search terms) are determined/selected by a used. The sets of search terms may include lists of research terms/items of interest, as obtained, selected or consolidated by a user. In the example show in FIG. 1, the search terms may include lists of such terms as, drugs, diseases, genes, formulations, and he like. In some embodiments, the search term list may be obtained from databases. Ion some embodiments, in this step, the user may choose search term(s) (also referred to herein as search item(s)) lists (sets) from various databases or individually selected by the user, for example, based on publications/manuscripts, etc. As non-limiting examples, a list (set) of drugs (search terms) may be obtained from databases, such as, drugbank.com (6000 drugs), FDA database (1900 drugs), commercially available FDA approved drugs (1900 drugs), list of kinase inhibitors from Selleckchem.com, and the like. As non-limiting examples, a list (set) of cancer types (search terms) can be obtained from the National Cancer Institute or AACR. As a non-limiting example, a list (set) of targetable genes (search terms) may be obtained from memorial Sloan Kettering Cancer Center (MSKCC) integrated mutation profiling of actionable cancer targets (IMPACT). In some embodiments, it is preferable that search terms lists include terms/words that have only one meaning to improve search results. For example, if a searched drug is also a neurotransmitter (for example, dopamine), it may skew the results, since it can appear in the search as both. To this aim, a specific named drug (such as a trademark name) may be used as a search term, instead of the generic drug. For example, in the case of injectable Dopamine, the trade name Intropin™ may be used to improve results. In some embodiments, the item list may include not only scientific terms (items), but any other suitable terms, such as, for example, but not limited to: countries, universities, authors, and the like. In some embodiments, a list of terms may also be extracted from papers utilizing suitable word document extractor tools, such as word-clouds generators.

As further shown in FIG. 1, in the second step (2), multiple hypotheses are generated using the hypothesis generator. The hypotheses generator may include a suitable processor (for example, of a suitable computer system), configured to generate the hypotheses. In some embodiments, using a combination text generator and according to sets of search terms of step 1 (i.e., the subject of interest), the user or the system can select what combination of terms would be used to generate hypotheses. According to some embodiments, based on the purpose or question of interest, the search can be structured as “one vs many” or “many vs many”. In some exemplary embodiments, for example, if the user is interested in a question such as: “what are the important genes in melanoma?” or “what is the most studied drug in Austria?” it is referred to herein as a “one vs many” structured search. In some exemplary embodiments, questions, such as, “which genes goes with which cancers?” or “what drugs goes with which side effects?” it is referred to herein as a “many vs many” structured search. In some embodiments, upon selecting the search structure and the sources of the lists, the hypothesis generator algorithm generates all possible word combinations from the lists into a new matrix, that can be in the form, for example, of a list (one vs many) or an arrayed matrix (many vs many).

Next, as shown in FIG. 1, in step 3, and automated literature search for the generated hypotheses can be performed. The automated search can be performed using, for example, a web scraper that can extract the number of publications/results per each generated hypothesis (i.e., combination of selected terms). In some embodiments, in this step, all (or any portion of) the generated hypotheses are automatically being searched, using, for example, a web crawler, on suitable databases. In some embodiments, the searchable databases are digital databases. In some embodiments, the databases are located on a remote server and are accessible over a network or internet. In some exemplary embodiments, as illustrated in FIG. 1, the searchable databases can include Google Scholar or PubMed. In order to get faster extraction of NOPs, it is possible to connect to the API of PubMed, such that, for example. 10000 results will take roughly 20 minutes instead of 160 minutes.

As further shown in FIG. 1, in the next step (4), the automated search results are retrieved, and the number of publications (NOP) of each searched hypothesis is extracted/determined. The NOP results are inserted into a NOP list or a NOP array matrix depending on the search structure. In some embodiments, the NOP may be correlated with the strength of a hypothesis, based on the assumption that in the scientific literature, true statements or hypotheses appear more (quantitatively) than false statements.

As shown in FIG. 1, in the next step (5), the results of the search (for example, NOP of hypothesis) may be graphically presented. In some embodiments, as illustrated in FIG. 1, the results may be presented as a color-coded hypotheses matrix, or any other suitable presentation form. In some embodiments, the NOP matrix may be visualized using color (shades) coding settings menu with adjustable thresholds of what may considered a “strong” hypothesis. The adjustable thresholds may include, for example, what is considered a reasonable hypothesis and what is considered not reasonable. For example, 0 publications may be marked as dark gray shade (originally red), 10 publications marked as brighter gray (originally orange) and over 20 publications as light gray (originally green). In some embodiments, the color or shades coding scale and the thresholds according to which the scale is presented, may be predetermined or determined by a user and adjusted at will. In the next step (6), the generated NOP matrix may be further sorted and the various hypotheses may be ranked within the initial matrix. In some embodiments, the NOP hypotheses matrix may be sorted in several different ways. In some exemplary embodiments, the matrix may be sorted by the highest value in each column or the highest sum of the cells in each column. In some embodiments, it is possible to sort column by clustering cells in the matrix, and normalize or weigh the matrix to have a ratio compared to the strongest hypothesis, as further detailed below.

As further shown in FIG. 1, at the next step (7), the prediction of novelty, feasibility and or reasonability of the generated hypotheses may be optionally be generated and presented. Further, optionally, in step 7, additional search term (variables) may be added to selected hypotheses (for example, to top ranked hypotheses). In some embodiments, adding new and relevant variables to selected hypothesis may be used to generate yet multiple new hypotheses. In some embodiments, optionally, this step can also include combining results of two separate searches into a new (third) search. In such embodiments, after the matrix is sorted in step 6, it may be modified to add search terms of interest, adding additional complexity to the previous generated/identified hypotheses. In some embodiments, it may then be possible to predict or extrapolate whether the additional variable is meaningful, for example, with respect to novelty. In some embodiments, the addition of a new search term into an existing matrix results in the creation of a new matrix, which may than be optionally overlaid or merged with the previous one for comparison.

According to some embodiments, at the final analysis output, the obtained results may be sorted, ranked and/or merged by the strongest hypothesis or with highest novelty potential and feasibility. The results may be visually presented to the user, with the initial subject of interest and present a color-coded map containing all of the quantitative NOP results from the multiple hypothesis searched, optionally merged with the additional search terms (variables), if used. In some embodiments, the result matrix thus represents a meta-analysis of the literature in a field of interest, optionally including ranking of potential novelty, reasonability and/or feasibility of unpublished (previously unknown) hypothesis. In some embodiments, further analysis of the matrix (for example, by using mathematical analysis), can propose even more hypotheses.

According to some embodiments, additionally or alternatively to graphical presentation, a user may choose a textual output of the hypotheses of interest.

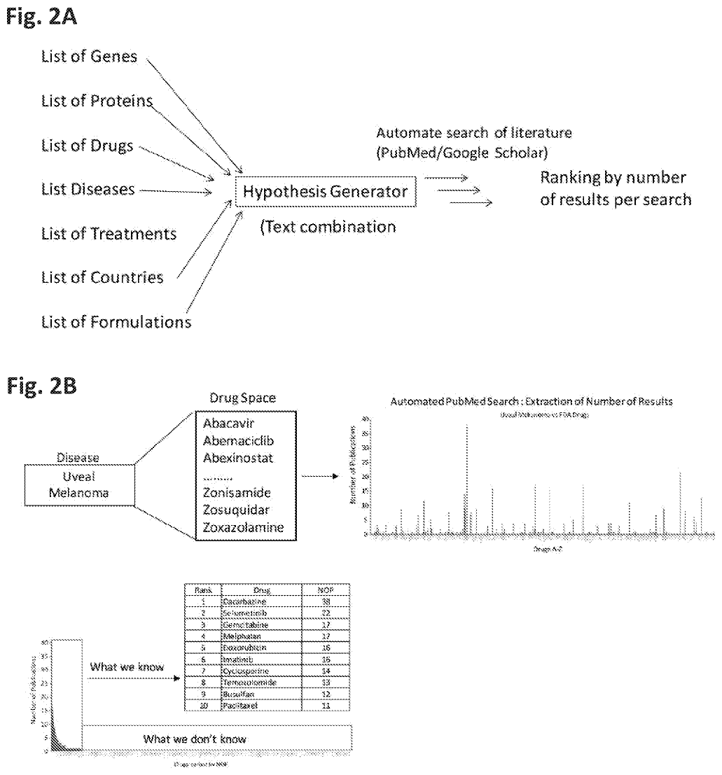

Reference is now made to FIGS. 2A-B, which exemplify steps 1-3 in the method for automated literature meta analysis, according to some embodiments. As shown in FIG. 2A, a set of search terms (such as list of genes, list of proteins, list of drugs, list of diseases, list of treatments, list of countries, list of formulations, etc.) is selected. The search terms are then used to generate respective hypotheses (combinations of search terms), which are then automatically searched on suitable databases (such as, for example, Pubmed, google scholar) and the obtained results are ranked by NOP of each searched hypothesis. FIG. 2B shows exemplary automatic search using 1800 FDA approved drugs (search terms) together with the rare disease uveal melanoma (search term). The generated hypotheses are presented in a graph matrix shown in the right hand column of FIG. 2B, which illustrates the relation between the drug name and the respective number of publications. The lower panel of FIG. 2B, shows another presentation of the results, which are sorted in a table based on the NOP of the respective drugs.

In some embodiments, as detailed herein, the search may be constructed as “one vs many”. In a meta-analysis of “one vs. many”, a major goal may be to find leads and get a sense of what is important in a certain field. In some embodiments, such a search is not necessarily for evaluating lack or holes in knowledge, but more for identifying the major important factors in said specific field. In some embodiments, the approach of ‘one vs many’ can further be used as a first step in analyzing ‘many vs. many’ searches, in order to screen out items that have no publications and therefore should be excluded from future searches in that specific field for the purpose of saving time and computation efforts. In some embodiments, using one vs many search can provide information regarding questions that are very hard to answer in a manual (non-automated) search. Example 2, presented herein below exemplifies a “one vs. many” structured search for the most important genes and drugs in uveal melanoma.

According to some embodiments, in a ‘many vs many’ structured search, the purpose is to look at multiple possible combinations and identify/detect larger publication landscape of combinations/hypotheses. Such a structured search can be used to show which hypotheses have been published together with ones that have not been published. In some embodiments, the reasoning or assumption that a proposed scientific hypothesis has no publications can be either that it may be obviously false and thus it makes no sense to test or publish it, or that it is potentially true but it has not yet been tested nor published.

According to some embodiments, the methods and systems disclosed herein can be easily used to identify and visualize novel hypotheses (i.e. hypotheses that were never published), which are both reasonable and feasible, by adding search variables to leading identified hypotheses. This is exemplified in example 4, herein below.

According to some embodiments, a scoring system may be assigned for the generated hypothesis, to indicate the novelty, feasibility and/or reasonability thereof. In some embodiments, in order to assign a scoring system for the generated hypothesis, a set of conditional statements may be used for the merged matrices. In some embodiments, a first step can include setting the respective thresholds (for example, similarly to the same way they are set for colorization/shading presentation). The thresholds are important to define what is potentially true and what is novel. A high threshold is defined as the number of publications that above it, it is indicative that the hypothesis is true or established. A medium threshold is used to describe the potential truth and can also be used for reasonability calculations.

According to some embodiments, a comparison matrix may be derived from a search matrix by generating a new search task with an additional string and layering together the original matrix with the new matrix side by side for comparison of hypotheses with or without one of the elements. In some embodiments, the allows the process of triangulation in the ranking algorithm.

According to some embodiments, for evaluating the novelty (N) parameter of a hypothesis, a numerical descriptor can be defined for an individual cell in the matrix (a single hypothesis) as N=Novelty. In this descriptor, only the new added concept/word in the merged comparison matrix (also called ‘var’ cell or the right cell) is looked at. If the NOP of the var=0 then N=2. If the NOP of var is between 1 to the medium threshold (set/determined by the user) then N=1. If the NOP of var is higher than the high-threshold value, then N=0.

According to some embodiments, the parameters of reasonability can be classified into three sub-criteria: Local reasonability (LR); Horizontal reasonability (HR) and vertical reasonability (VR). In some embodiments, the Horizontal reasonability (HR) and/or vertical reasonability (VR) may be extended.

According to some embodiments, a Local Reasonability (LR) descriptor is used to examine the respective cell from the initial matrix (the left cell, or LC). The score of LC is the LR. If LC>high threshold, then LR=2, If med<LC<high then LR=1. If LC<med threshold then LR=0.

According to some embodiments, a Horizontal Reasonability (HR) descriptor reads the ‘var cells’ or right cells of the new matrix in the same row or ‘the horizontal’ setting. These cells are also named HorVar (horizontal var) and the scoring of the horizontal cell is HR. IF HorVar>high threshold, then HR=2, IF med<HorVar<high then HR=1, IF HorVar<med threshold then HR=0

According to some embodiments, a vertical Reasonability (VR), is the same as HR but in vertical direction. The VR descriptor looks at the ‘var cells’ or right cells of the new matrix in the same column or ‘the vertical’. These cells are also named VerVar (vertical var) and the scoring of vertical cells—VR.

According to some embodiments, HR and VR can be considered also as feasibility descriptors, as they add to the reasonability of the hypothesis through what is possible in adjacent hypotheses in the same narrow field, which can indicate how easy or hard the execution of the hypothesis will be.

According to some embodiments, HR and VR can be extended beyond the basic comparison matrix to include other (partial or all) relevant searches. For example, if a basic search matrix includes 5 drugs (vertical) and 5 cancers (horizontal), and the variable (Var) is ‘Radiotherapy’, the extended HR (also referred to herein as “total HR” or “THR”) reflects all results from ‘Radiotherapy-Doxorubicin (drug)’ with all the diseases and not a specific cancer. The extended VR (also referred to herein as “total VR” or “TVR”) reflects the results from ‘Radiotherapy-Melanoma (Cancer)’ with all the possible drugs and not a specific drug.

According to some embodiments, the parameters of reasonability can be classified into: Local reasonability (LR); Horizontal reasonability (HR), vertical reasonability (VR). Extended horizontal reasonability (THR), Extended vertical reasonability (TVR), or any combinations thereof. Each possibility is a separate embodiment.

According to some embodiments, when hypotheses are ranked by N, LR, HR and/or VR (and/or in some cases also by THR or TVR), various elements about the hypothesis matrix can be deduced, including, for example, what are the leading true and validated hypothesis, what are unpublished but highly potential true hypothesis, and what are novel and with lower potential to be true.

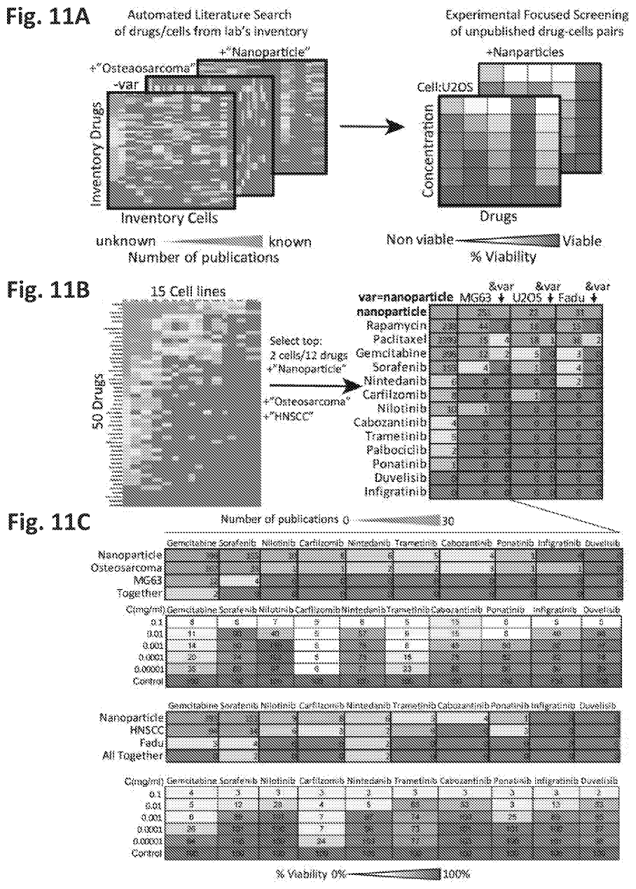

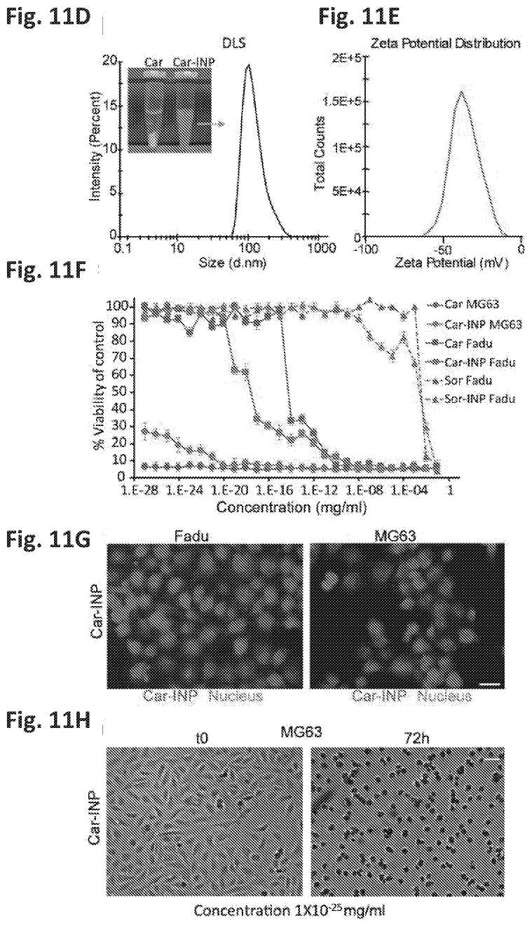

According to some embodiments, an important factor for literature review and scientific research in general, is to know which hypothesis is emerging as an important truth or is trending in a scientific field. In some embodiments, it may be regarded as another aspect of novelty. To this aim, in some embodiments, the methods disclosed herein may further include a step of extracting of the number of publications per year. As demonstrated in FIGS. 11A-C the yearly publications of five different cancers together with six different variables search terms are presented. The number of publications (NOP) was normalized to the highest NOP of the specific cancer. This allows identifying, for example, what are the emerging new hypotheses of the last X (for example, 5) years. In the examples presented in FIGS. 11A-C, the hypotheses include treatments based on PD-1 and CTLA-4 in all cancers, doxorubicin for chondrosarcoma and trametinib for thyroid cancer.

According to some embodiments, the systems methods disclosed herein may further be utilized to visualize the hypotheses temporal landscape, i.e., the emergence or decline of biomedical hypotheses. In some embodiments, the methods thus allow to automatically identify the most trending hypotheses and compare them to steady or declining hypotheses.

According to some embodiments, the methods disclosed herein may further be utilized to visualize the hypotheses geographical landscape. i.e., the geographical distribution of biomedical hypotheses. In some embodiments, the methods allow to automatically identify the trending hypotheses based on the geographical origin of the data used for the generation of the hypotheses.

According to some embodiments, there are provided methods and systems for visualization of the temporal landscape, or in other words, the rise and fall of biomedical hypotheses. This can be used to automatically identify the most trending hypotheses and compare them to steady or declining hypotheses.

According to some embodiments, there is provided a computer implemented method for generation and ranking of hypotheses, by automated literature meta-analysis, on one or more sets of search terms, the method includes one or more of the steps of:
a. obtaining one or more sets of two or more search terms;
b. generating multiple hypotheses, based on a selected combination of the search terms;
c. performing a search for the generated hypotheses on one or more suitable databases stored on a server, to determine the number of publications (NOP) for each generated hypothesis;
d. generating a matrix of the NOP of one or more selected generated hypotheses;
e. sorting the NOP matrix of the one or more selected generated hypotheses, based on one or more sorting parameters; and
f. ranking the selected generated hypotheses based on the NOP matrix, wherein the ranking is indicative of the degree of novelty and/or degree of feasibility and/or degree of reasonability of the selected generated hypothesis.

According to some embodiments, the method may further include a step of performing an additional search using a second set of search terms or search variables on the sorted NOP matrix of the one or more selected generated hypotheses. In some embodiments, this step further includes the formation of a comparison matrix, between the first search with the first set of search terms, and the second search with the second set of search terms.

In some embodiments, the method may further include a step of presenting one or more of: the matrix of the NOP, the sorted matrix of the NOP, normalized NOP, color coded NOP, merged NOP matrices, the ranking of the selected generated hypotheses, or any combination thereof. Each possibility is a separate embodiment.

According to some embodiments, the hypothesis may be a scientific hypothesis, an experimental finding, medical procedure(s), a general question, and the like, or any combination thereof.

According to some embodiments, each search term may be selected from: a word, list of words, a sentence, a generic term, a question, and the like, or any combination thereof. Each possibility is a separate embodiment. Exemplary search terms may include such terms as, but not limited to: list of chemical or biological substances, list of molecules, list of genes, list of proteins, list of drugs, list of administration routes, list of carriers, list of formulations, list of disease, list of treatments, list of institutions, list of researchers, list of countries, and the like.

In some embodiments, the search terms and/or search sets may be selected by a user or may be provided from a respective database.

According to some embodiments, the selected combination of the search may be structured as “one vs. many” (“one versus many”) and/or “many vs. many” (“many versus many”, or both.

According to some embodiments, the search may be performed using a suitable web crawler, web scraper, general automated search tool, and the like, or combinations thereof.

In some embodiments, the databases may be selected from PubMed, Google Scholar, Embase, clinicaltrials.gov, and Semantic Scholars, and the like, or any combinations thereof. In some embodiments, the databases are electronic databases. In some embodiments, the databases are stored on a server. In some embodiments, the server is located at a remote location and may be accessed via a network (such as, World Wide Web).

In some embodiments, the NOP matrix may be visualized using a visual coding having adjustable threshold, based on the visualization parameters, such as, coloring or shading. In some embodiments, the NOP matrix may be visualized by any suitable means, including, for example, text and graphics.

According to some embodiments, the degree of novelty, feasibility and/or reasonability may be determined based on an adjustable threshold. In some embodiments, the adjustable threshold may be number of publications. In some embodiments, more than one type of threshold may be determined, for example, high, medium or low threshold. In some embodiments, the adjustable threshold may be user defined, or automatically preset.

In some embodiments, the methods disclosed herein may further include determining and presenting a numerical score based on the ranking of the hypothesis, which is indicative of the hypothesis, with respect to its strength, as determined based on novelty, reasonability and/or feasibility. Each possibility is a separate embodiment.

According to some embodiments, there is provided a system comprising a processor configured to execute a method for automatic generation and ranking of hypotheses, by automated literature meta-analysis, as disclosed herein. In some embodiments, the system may further include a user interface, a display unit, a communication unit, or any combination thereof.

According to some embodiments, there is provided a non-transitory, tangible computer-readable media having computer-executable instructions for performing the method for hypothesis generation and automated literature meta analysis searches, by running a software program on a computer, the computer operating under an operating system, the method including issuing instructions from the software program.

According to some embodiments, the systems and methods disclosed herein can be used as a hybrid of ‘hypothesis driven science’ and high throughput screening (HTS). In some embodiments, they utilize automation to generate multiple hypotheses.

According to some embodiments, and as disclosed herein, the utilizing the systems and methods disclosed herein it is possible to look at unpublished hypotheses and evaluate their reasonability and novelty by comparing publications between different elements in the hypotheses.

In some embodiments, the reasonability and novelty as used herein imply that they represent an anti-correlated duality. In some embodiments, the most reasonable idea is usually a well-known idea, which is the least novel, and the more novel idea is the one that has the least obvious reasonability. According to some embodiments, the reasonability of known parts of complex hypotheses can be summed and consequently infer the reasonability of the entire hypothesis based thereon.

According to some embodiments, as detailed and exemplified herein, for hypotheses with three different elements, a triangulation method may be used for ranking various relationships between various variables, such as, for example, but not limited to: cancer-drug-radiation combinations, cancer-drug-nanoparticle, biomaterials-targets-disease, by reasonability and novelty.

In some embodiments, a triangulation may at least partially utilize or at least partially be based on extended reasonability (such as, extended vertical reasonability and/or extended horizontal reasonability).

According to some embodiments, as exemplified herein, the systems and methods disclosed herein may be used to propose novel experiments based on lists of available reagents. For example, as demonstrated in Example 8 below herein, the systems and methods were used to perform focused screening on 20 drugs that were not tested in osteosarcoma and head and neck cancer. Accordingly, carfilzomib, a drug used in multiple myeloma as a highly potent compound in osteosarcoma was identified.

According to some embodiments, the systems and methods may further utilize temporal and/or geographical data to generate corresponding temporal and/or geographic distribution of biomedical hypotheses. Such temporal and/or geographical distribution may be used in the field of meta-science, and may maximize research quality.

According to some embodiments, the systems and methods disclosed herein may be used for identifying the temporal occurrence of hypotheses. This enables of identification of trending hypotheses and decreasing hypotheses over time.

According to some embodiments, the systems and methods disclosed herein may be used for identifying the geographic distribution of hypotheses.

According to some embodiments, the methods and systems disclosed herein may be used for identifying type and/or optimal formulation of a drug, such, a small molecule drug.

According to some embodiments, the methods and systems disclosed herein may be used for identifying the most reasonable biomarkers for a disease condition, such as, for example, cancer.
1. A computer implemented method for identifying optimal formulation of a small molecule drug.
2. A computer implemented method for identifying the geographic distribution of hypotheses.

A computer implemented method for identifying the most reasonable unpublished biomarkers of disease such as cancer.

According to some embodiments, the methods and systems disclosed herein may further be used to identify and/or determine a treatment or treatment regime for specific disease, such as, for example COVID-19 infection.

According to some embodiments, the methods and systems disclosed herein may further be used to identify and determine a high resolution combination therapy (HRCT) treatment regime. In some embodiments, the HRCT can be individualized (personalized) to specific patients, such as, cancer patients.

In some embodiments, due to the ability of the methods and systems disclosed herein to perform automated literature meta analysis searches and to identify and rank hypotheses, it can also be used to identify and determine complicated treatment regime that can be specifically tailored to a specific patient.

According to some embodiments, the provided systems and methods can automatically integrate hundreds of scientific findings into a personalized, complex and highly detailed treatment plan while ranking the elements of the plan by novelty/risk, reasonability and feasibility.

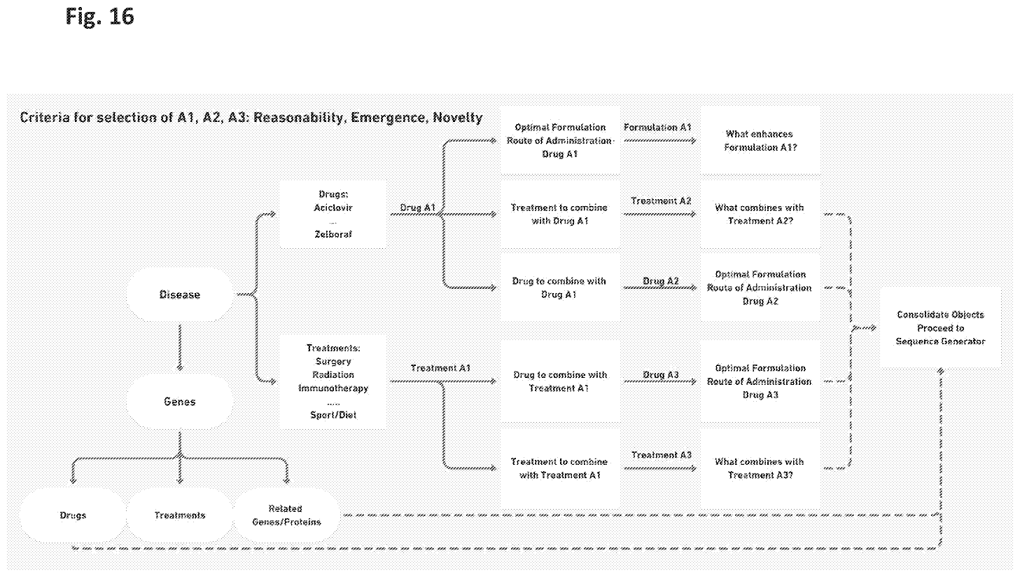

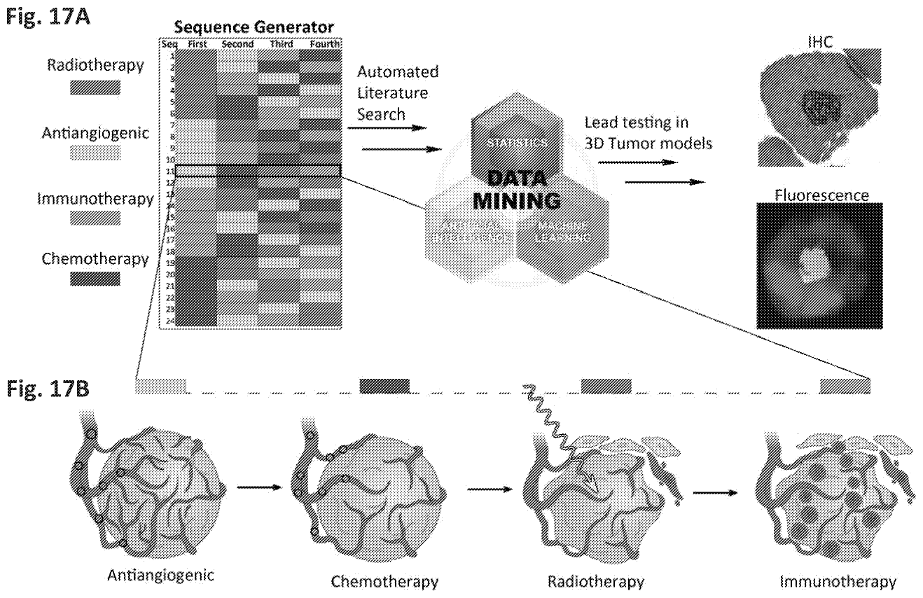

According to some embodiments, the method disclosed herein can be used as building block in a framework for high-resolution combination therapy (HRCT). Reference is now made to FIG. 16, which illustrates an exemplary plan to design/determine combination treatment plan. Starting with a specific disease, the methods disclosed herein are used to find the most common or most reasonable single drug to be used for that disease. Then, ALMA is re-applied to find, for example, the best formulation for that specific drug, what other single drug is most reasonable to combine with the first drug, as well as other suitable treatment modalities (such as, radiation, immunotherapy, etc.) to be combined therewith. This search is then further applied to the second drug/treatment/formulation. This recursive procedure can be repeated until it reaches the complexity level defined by the user (for example, how many elements make it unfeasible). In some embodiments, if genetic information regarding the patient is available, the search algorithm (ALMA), can be applied to the specific mutated genes in the same manner. Once all the various elements are collected, they can go through a sequence generator (as illustrated in FIG. 17A). After the elements are gathered and the various relationships thereof is determined, in order to generate a suitable sequence, a sequence generator can try possible sequence until it adds evidence for an estimated sequence. The different sequences are automatically searched (for example, online no suitable databases), to find an optimal order by collecting and adding together pairs of information. In some embodiments, a sequence generator is a word combination generator that can incorporate words that are temporally descriptive, such as, “before”, “after”, “weekly”, “daily”, “biweekly”, and the like.

According to some embodiments, generating HRCT using the methods disclosed herein is advantageous, since when generating a suitable HRCT, several inherent conceptual limitations in proposing highly complex treatment plans make this endeavor highly challenging. Conceptually, one would need to acknowledge that with increasing complexity, traditional controls are practically impossible. If, for example a combinatory treatment is a suggested plan of four drugs given sequentially at specific times. Theoretically, a fair comparison of the proposed sequence will be against all possible permutations of that sequence (4!=4*3*2*1=24) and should compare twenty-four different sequences with the exact timing. If one wishes to consider the timing as a variable, then the level of complexity of controls will be almost infinite. Thus, such limitation should be addressed by comparing to gold standards. A second crucial limitation is feasibility and compliance.

In some embodiments, when combining two or more drugs that work in synergy, such compounds may often exhibit vastly different chemical properties (e.g., size, charge, lipophilicity, and stability), hindering co-localization within tumor tissues in a timely manner. In addition, the emergence of even more toxic adverse side effects, due to inhibiting two or more pathway effectors simultaneously is often limiting the dose of combination therapy, which in turn limit the efficacy. Therefore, despite the strong rationale for their clinical testing, many patients do not show durable responses to these therapeutic strategies, because severe side-effects prohibit increasing the dose to allow sufficient exposure of the tumor cells to the drug combination. Additionally, delivery means of the drugs also complicate the treatment. Thus, by utilizing the methods disclosed herein, as well as cheminformatic tools, in addition to the data mining tools can be used in order to maximize efficient formulation process of any drug structure. In this manner it may be possible to optimize every single aspect of the treatment, from the type of drug regiments down to the molecular level of the formulation. The drugs identified are matched to the disease and then the formulation is matched to the drug and the disease.

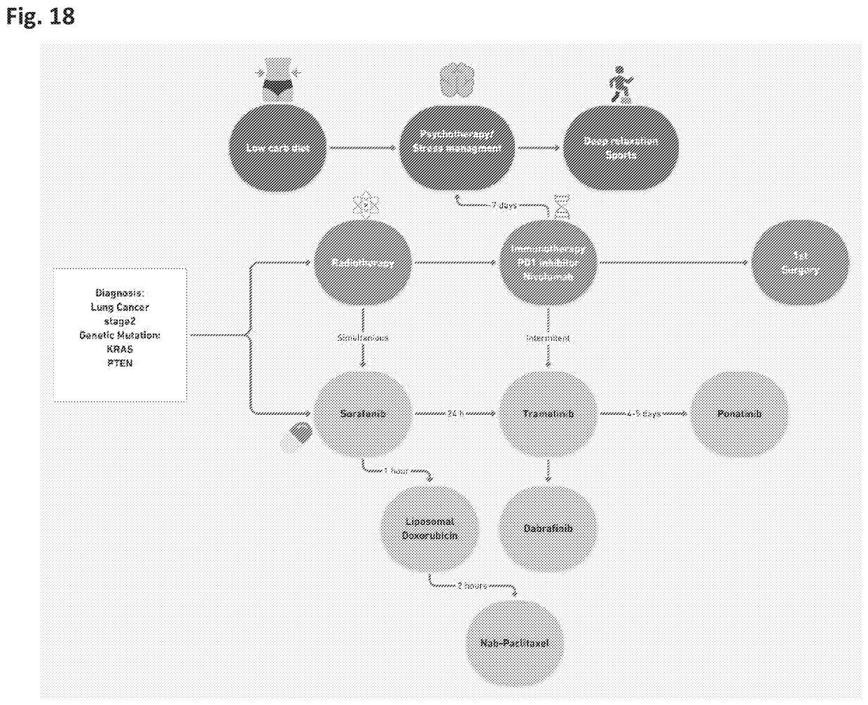

According to some exemplary embodiments, as further exemplified in Example 7, below, an example for the HRCT generation workflow can include, questions such as, what is the top drug for a specific mutation, what other drug goes with the identified first drug, what additional treatment goes with the identified drugs, what goes with the identified additional treatment, and so on. The results of such detailed treatment regime are presented in FIG. 18, which lists the various treatments and intervention procedures, as well as their sequence and temporal distribution.

According to some embodiments, there is provided a computer implemented method for determining a personalized high resolution treatment regime of a patient afflicted with a disease, the method may include one or more of the steps of:
obtaining a set of two or more search terms related to the disease of the patient;
generating multiple hypotheses related to treatment of the disease, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters;
ranking the selected generated hypotheses based on the NOP matrix, wherein the ranking is indicative of the degree of novelty and/or degree of feasibility and/or degree of reasonability of the selected generated hypothesis, to determine a first treatment;
repeating the search for one or more times with search terms related to the disease and/or the first treatment, to determine an additional one or more treatments; and
determining, based on the identified treatments, a personalized treatment regime for said patient.

According to some embodiments, there is provided a computer implemented method for determining a personalized high resolution treatment regime of a patient afflicted with a disease, the method may include one or more of the steps of:
obtaining two or more sets of search terms;
generating combinations of search terms from the sets, each combination corresponding to a hypothesis related to treatment of the disease;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of the degree of novelty and/or degree of feasibility and/or degree of reasonability of the hypotheses, to determine a first treatment;
repeating the search for one or more times with search terms related to the disease and/or the first treatment, to determine an additional one or more treatments; and
determining, based on the identified treatments, a personalized treatment regime for said patient.

According to some embodiments, the treatment is a combination therapy. According to some embodiments the patient is a cancer patient.

According to some embodiments the first treatment and/or the one or more additional treatments are selected from: a drug, an immunotherapy, a surgical procedure, radiotherapy, chemotherapy, psychotherapy, lifestyle therapy, or any combination thereof.

According to some embodiments the treatment regime may further include a spatial distribution sequence of the first and/or additional treatment.

According to some embodiments, there is provided a non-transitory, tangible computer-readable media having computer-executable instructions for performing the method for determining a personalized high resolution treatment regime of a patient afflicted with a disease.

According to some embodiments, the methods disclosed herein are computer implemented methods.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1—Using ALMA to Identify New Hypotheses

In this example, the proto-oncogene BRAF is used as one search term and cancer types are used as another search term(s). The suggested hypotheses were generated using text combinations that involve all known cancer types together with the BRAF gene (i.e., “gene, disease” search terms).

An automated search of all hypotheses in the list was performed and the number of results (or number of publications per search) of each item in the list was extracted from the search. The list (matrix) was sorted by number of publications (NOP) so that the strongest hypothesis is at the top. The results are presented in FIG. 3. In this example, melanoma is the cancer that has the most association with BRAF, followed by lung cancer.

Thereafter, another vertical automated search is performed on BRAF and all known drugs (gene, drug). The second list of hypotheses is generated, searched and sorted. In this exemplary search, the most common drugs associated with BRAF were vemurafenib, dabrafenib and trametinib and their combination.

Then, a third list of hypotheses was generated by combining the two previous searches: all BRAF related cancers together with BRAF related drugs (gene, disease, drug). An automated search of the hypotheses list and extraction of NOP yielded a disease-drug matrix that included the number of publications per drug-disease association with BRAF focus.

Further, the strongest hypothesis can also be modified to add text variables to evaluate further, what is scientifically known and unknown. For example, the variables could be, clinical trials, novel therapeutic combinations such as immunotherapy (nivolumab is used in the example), drugs with similar mechanism of action (cobimetinib and vemurafenib in our example) etc. One possible presentation of the result is shown in FIG. 3, which shows a color (shading) coded map/matrix of what is scientifically known (light-bright gray (originally green-yellow) and what is unknown (dark gray (originally red)). Based on the presented results, high potential discoveries in the dark (red) area that are in close proximity to the strongest hypothesis which is the one with the most publications can be derived and identified. Such high potential hypotheses include, for example, treating BRAF driven non-small cell lung cancer with cobimetininb and vemurafeni combination.

To simplify presentation and to consider the limited space, Vemurafenib, cobimetinib, clinical trial, nivolumab single searches were excluded from the matrix.

### Example 2—“One Vs. Many” Structured Search Using ALMA

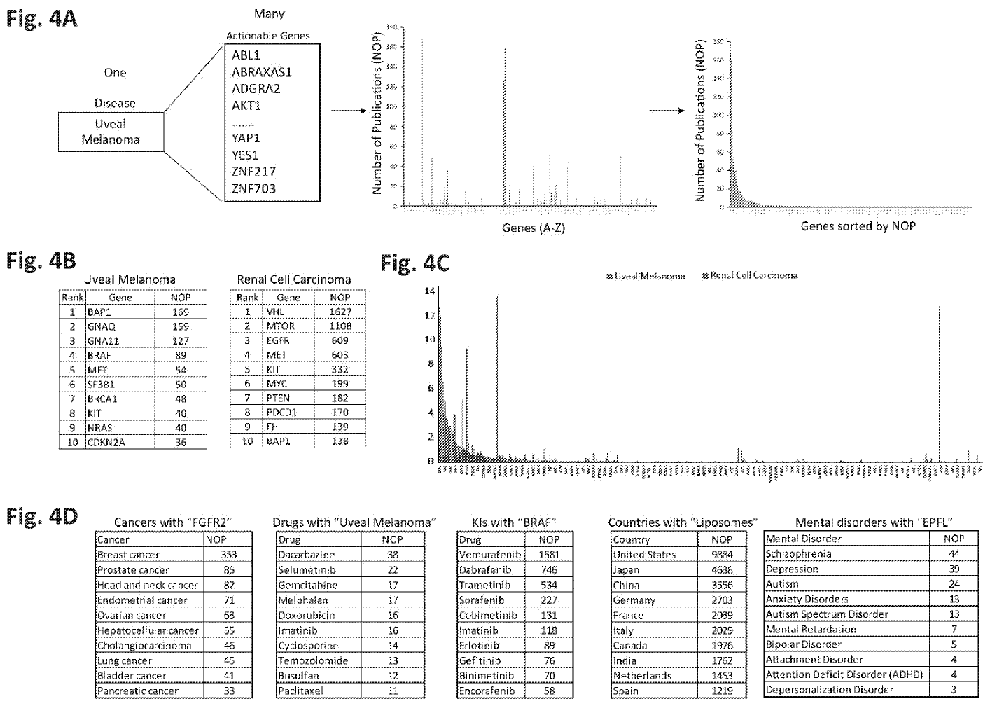

In this example, ALMA was used to search for the most important genes and drugs in uveal melanoma (a rare cancer). The search was focused for the list of targetable genes (400 genes) and thus generated 400 search strings of the genes with uveal melanoma. Results are shown in FIG. 4A—as can be seen, from about 400 targetable genes, only a third has any publication with uveal melanoma (UM) in title or abstract and less than 10% of these genes has more than 10 publications in this disease. The top 10 studied genes in UM are shown in FIG. 4B. Comparing the same search for renal cell carcinoma (a form of kidney cancer), shows a very different pattern of publications, as can be seen in FIGS. 4B-C.

The approach of ‘one vs many’ can further be used as a first step for analyzing ‘many vs. many’, in order to screen out items that have no publications and therefore should be excluded from future searches in that specific field for the purpose of saving time and computation efforts. A similar manual search by a human takes several hours and even days whereas the automated search takes minutes.

In addition, using “one vs many” search can provide information regarding questions that are very hard to answer in a manual (non-automated) search. This is illustrated in FIG. 4D, which presents exemplary automated results regarding questions, such as, ‘what are the top ten most studied mental disorders in Ecole polytechnique fédérale de Lausanne (EPFL) institute?’ or ‘which countries lead the research on liposomes?’ that would otherwise be very difficult to answer with standard non automated (manual) search tools.

### Example 3—‘Many Vs Many’ Structured Search Using ALMA

In this example, ALMA is applied in a ‘Many vs Many’ search, which includes, Hypotheses NOP (number of publications) matrix sorting, identification of leads and holes in a scientific field.

In the ‘many vs many’ search structure, the purpose is to look at multiple possible combinations and identify/detect larger publication landscape of combinations. Such a structured search can be used to show which hypotheses have been published together with ones that have not been published. The reasoning that a proposed scientific hypothesis has no publications can be either that it's obviously false and it makes no sense to test or publish it, or that it is potentially true but it has not been tested nor published yet.

In this example, it was evaluated if one can know which hypothesis is potentially true but never tested. To this aim, sorting the respective matrix would cluster together strong hypotheses and compare them to weaker hypotheses.

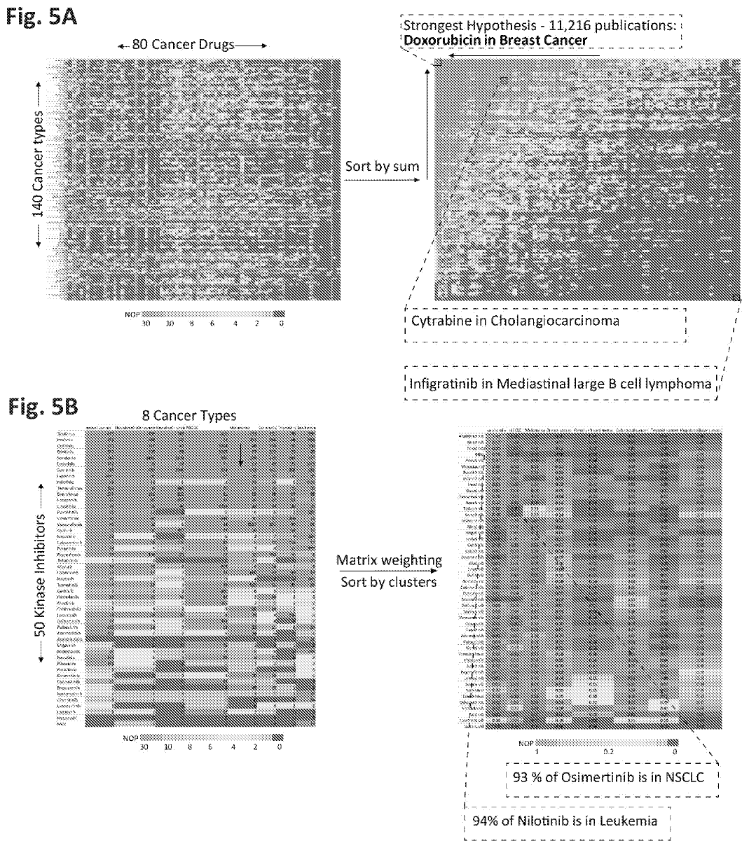

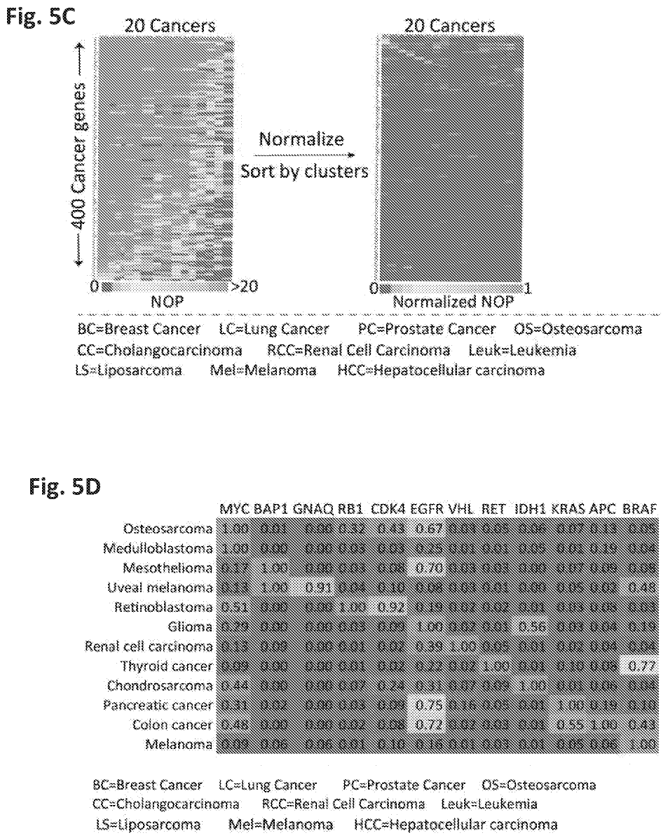

As an example, ALMA was applied to generate a hypothesis Matrix of 140 different cancer types together with 80 cancer drugs to see which drugs were used with which cancers (FIGS. 5A-B). The results yielded a matrix of about 11,200 different drug-cancer combinations in which each cell of the matrix array contains the NOP (extracted from PubMed's API). This matrix was automatically generated. The matrix was colored-coded and sorted by the highest sum of columns (FIG. 5A)—from left to right such that the strongest hypothesis is in the top left (which in this settings is the drug doxorubicin in breast cancer, having more than 11000 identified publications). According to a basic premise, it is reasonable to assume that doxorubicin is used/studied in breast cancer. The results further present/hint that some combinations were not studied or published (NOP=0). Interesting to note that that some hypotheses that are closer to the strongest hypothesis can be considered as more reasonable than hypotheses that are farther from the strongest hypothesis. For example, as shown in FIG. 5A, a drug-cancer combination ‘Cytarabine in cholangiocarcinoma’ (a type of liver cancer) was never published (NOP), even though it is a broad chemotherapy (a non-specific anti-metabolite chemotherapy), useful for many cancers. In contrast, the hypothesis of ‘infigratinib in Mediastinal large B cell lymphoma’ represented a targeted personalized medicine for solid tumors with active FGFR signaling which is not common in lymphomas. Such comparisons can thus allow to find ‘holes’ in the matrix and to perform an initial estimation whether an unknown hypothesis is reasonable or not by its proximity to known hypothesis. Further, if focusing on understanding and evaluating the leading hypotheses, the matrix can be sorted by cell clustering, as can be seen in FIG. 5B. ALMA was applied to generate a matrix of 50 FDA approved kinase inhibitors with eighth different cancer types (total of 400 hypotheses). A clustering algorithm was used to normalize each column or row in a matrix by its highest value and then apply a cell-size sorting process. For example, the matrix was normalized horizontally (by highest NOP), so that for each drug there is only one major cancer that has a normalized nNOP=1. The clustering algorithm was used to sort the normalized matrix using a sensitivity threshold input from the user for optimal clustering. In the example shown, clusters of the top 10% were selected by using a threshold of 0.9 so that every nNOP below 0.9 was sorted to different clusters. As can be seen in FIG. 5B, the drugs are clustered in groups by their cancer indication which perfectly matches data reported in the literature (“REF”). Thus, the drugs clustered in groups by their indication clearly show the personalized nature of these drugs as most of them have only one type of indication. The data was validated with the major indications reported, for example, in drugbank.ca. Without the need to review any publication, the user may be informed about the kinase inhibitors and their indications and classify them by disease. Further, it can be observed that some drugs at the bottom of the matrix are used in several cancers, which can either indicated that they act as multi-kinase inhibitors (inhibit many kinases) or that their target kinase is expressed in many cancers.

Additionally, a search matrix was generated to match the KIs with their major target kinases. No false negatives were found and only two false positives out of 50 inhibitors and 30 kinases. One false positive was the group of MEK inhibitors that were matched to BRAF as well as MEK (0.9 and 1 respectively). This can be explained by the fact that BRAFV600E driven melanoma is treated exclusively with a combination of MEK and BRAF inhibitors and thus MEK inhibitors and BRAF are mostly mentioned together. The other false positive was MTOR which was high in many multi-kinase inhibitors such as sorafenib, sunitinib, and pazopanib which are known to have a MTOR as compensatory pathway. It was next sought to use ALMA to explore the genes and cancer space and identify the most studied genes for different cancers automatically. To this end, a search matrix which included 400 actionable genes from the MSK-IMPACT list vs 20 cancer types was generated. The results are shown in FIG. 5C. The matrix was then normalized per cancer (horizontally) so that each cancer has only one gene (nNOP=1). The matrix was then sorted to clusters to aggregate cancers with the same top gene together. A focused representation of 12 cancers with their top studied genes is presented in FIG. 5D. As shown in FIG. 5D, it is clear that every cancer has a unique genetic literature landscape. The results obtained with ALMA were cross validated with the literature, and indeed, from the list of 400 genes, Osteosarcoma and Medulloblastoma are mostly studied with MYC, melanoma with BRAF, Mesothelioma and uveal melanoma with BAP1, and Renal cell carcinoma with VHL. In addition, it is noted that EGFR is studied in many cancers but only in glioma it is the most studied gene.

### Example 4—Prediction of Novelty and Feasibility: Merging Matrices, Overlay-Novelty Identifier

As detailed above, the methods disclosed herein can easily identify and visualize novel hypotheses (never published) that are both reasonable and feasible, by adding variables to leading hypotheses.

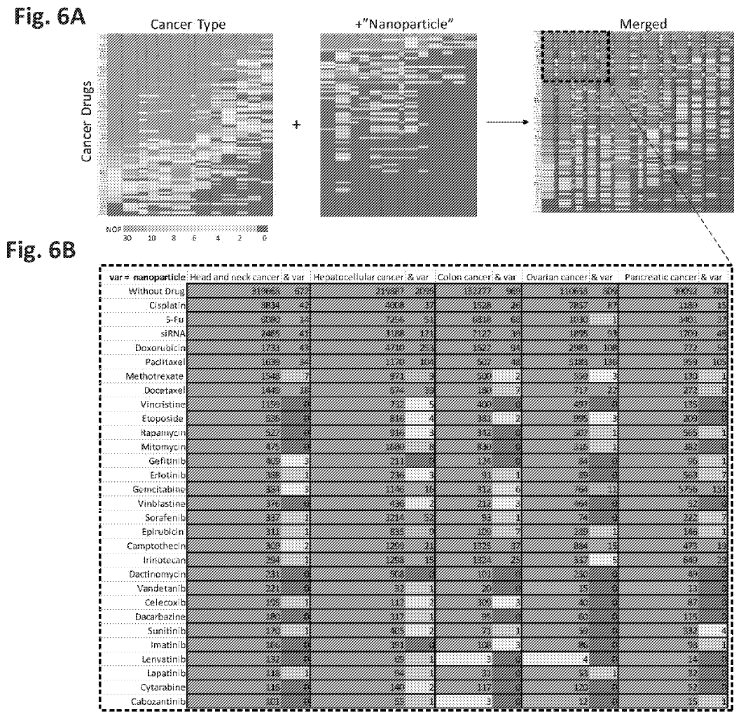

In this example, this approach is used to identify novel hypotheses in the field of cancer nanomedicine. To this end, ALMA was applied to generate a matrix of cancer drugs vs cancer types, which is then sorted by sum (as shown FIG. 6A). To the existing matrices, various search terms (variables) are added, and automatic searches can be run/performed on the new matrix. This feature was used to add to the drug-cancer matrix a text variable search term of the string “nanoparticle”, which is the most common word used in nanomedicine. This yielded a new matrix with fewer total publications. The two matrices were then merged to visualize the difference between them. As can be seen in FIG. 6B, if the focus is on strong hypothesis, while comparing the NOP with and without the new variable (i.e., the word “nanoparticle”) it can be relatively easily identified which hypothesis is novel and reasonable. Dark (red) cells next to brighter (green) cells are novel and reasonable, whereas bright (green) cells next to bright (green) cells are reasonable but are not novel (as the NOP is not 0). For example, the drug vincristine in head and neck cancer is published more than 1000 times without nanoparticles and 0 times with nanoparticles, which according to the premise, makes it a novel and a reasonable hypothesis. On the horizontal row of vincristine, it is also possible to see that vincristine nanoparticles were published on liver cancer, which makes vincristine nanoparticle feasible and the hypothesis of: vincristine, nanoparticle and head and neck cancer is considered novel, reasonable and feasible. Accordingly, the hypothesis can be formulated as “Vincristine loaded nanoparticles for head and neck cancer”. However, if a drug has never been published with nanoparticles, this may render it not feasible (for various reasons), as is the case with dactinomycin which has 0 publications with nanoparticles. Thus, such hypothesis (with dactinomycin) makes is highly novel (NOP=0), reasonable but the feasibility thereof is unknown. In contrast, it can be seen that paclitaxel has been published with nanoparticles in all cancers, rendering it highly feasible but not novel (NOP larger than 0).

### Example 5—Finding Novelty in Personalized Cancer Nanomedicine

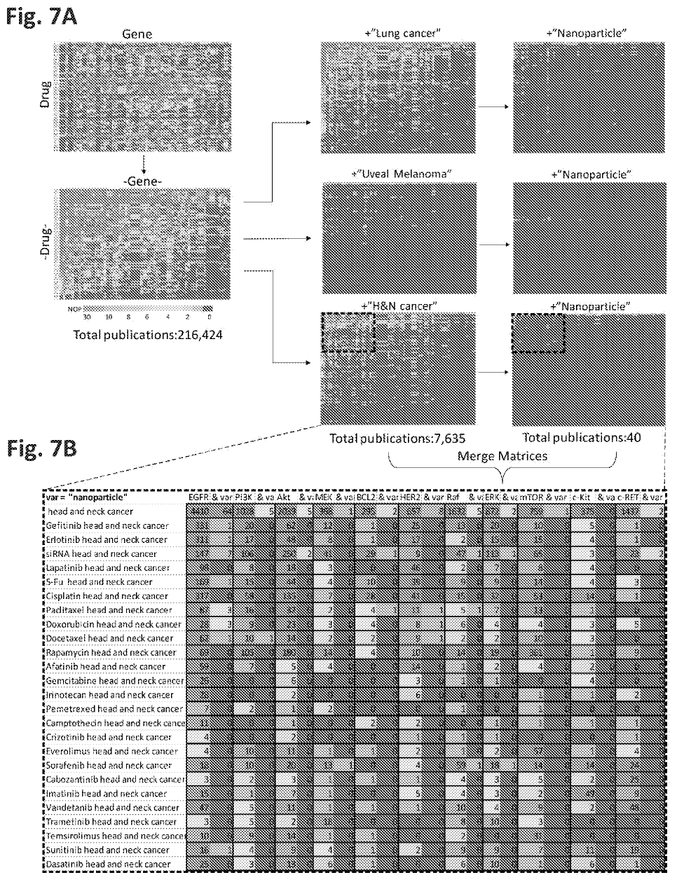

As another example, ALMA was applied to find novelty in personalized cancer medicine (FIGS. 7A-7B). This field is based on genetics of a tumor matching a drug loaded in nanoparticles. A drug-gene matrix was generated and sorted by sum. Preparation of the sorted Hypotheses matrix structured as: genes/drugs/and a cancer type followed by “nanoparticle”. The merged matrix contains the NOPs of all the cancer-drug combinations with and without the variable (var) “nanoparticle” side by side. Thereafter, different cancers of interest were added, followed by the addition of the search term (word) “nanoparticle”, as shown in FIG. 7A. The matrices were merged and the strong hypotheses of the first matrix (FIG. 7B) were scanned. The enlarged section in FIG. 7B shows the strongest cancers/drugs hypotheses. Numbers are NOPs of hypotheses. Dark gray (originally Red) indicates 0 publications and lighter gray (originally green) indicates more than 20 publications. Dark (Red) cells next to lighter gray (green) cells indicates of a hypothesis that is novel (never been published) but should be reasonable. If there are lighter gray (green) ‘&var’ cells in the row of that hypothesis then it is also feasible.

As can be seen, most common genes in head and neck cancer are EGFR, PI3K and AKT. Most nanoparticle containing papers focus on EGFR. Thus, it is possible to show that a gene-drug combination in a cancer can be personalized and checked if it is novel, reasonable and feasible. For example, for mTOR and c-KIT it can be seen that they have been mentioned 759 and 375 times, respectively, with head and neck cancer, but never tested in the context of a drug-nanoparticle. Thus, drugs having the highest value, such as Rapamycin and Imatinib for mTOR and c-KIT, respectively may be selected.

### Example 6—Quantifying or Scoring Novelty, Reasonability and/or Feasibility

As detailed above, in order to assign a scoring system for the generated hypothesis, a set of conditional statements may be used for the merged matrices. The first step is to set the respective thresholds (for example, similarly to the same way they are set for colorization/shading presentation). The thresholds are important to define what is potentially true and what is novel. A high threshold is the number of papers/publications that above it is indicative that the hypothesis is true or established (in the shading it is brighter gray (colorization it is a green color)). A medium threshold is important to describe the potential truth and can also be used for reasonability calculations.

For evaluating the novelty parameter of a hypothesis, a numerical descriptor is defined for an individual cell in the matrix (a single hypothesis) as N=Novelty:

In this descriptor, only looking at the new added concept/word in the merged comparison matrix (also called ‘var’ cell or the right cell). If var=0 then N=2. If var is between 1 to the medium threshold (set by user) then N=1. If var>high value then N=0.

The parameter of reasonability can be classified into 3 sub-criteria:

1. LR=Local Reasonability.

This descriptor examines the cell from the initial matrix (the left cell, or LC). The score of LC is the LR. If LC>high then LR=2, If med<LC<high then LR=1. If LC<med then LR=0

2. HR=Horizontal Reasonability.

This descriptor reads the ‘var cells’ or right cells of the new matrix in the same row or ‘the horizontal’ setting. These cells are also named HorVar (horizontal var) and the scoring of horizontal cells—HR.

IF HorVar>high then HR=2, IF med<HorVar<high then HR=1. if HorVar<med then HR=0

3. VR=vertical Reasonability. (same as HR but vertical)

This descriptor looks at the ‘var cells’ or right cells of the new matrix in the same column or ‘the vertical’. These cells are also named VerVar (vertical var) and the scoring of vertical cells—VR.

The HR and VR may further be extended. The extended HR and VR descriptors (Total HR (or THR) and Total VR (TVR)) may be formulated as follows: the HR and VR can be extended outside of the NOP matrix so that instead of or in addition to looking only in the vertical and horizontal cells in the matrix, it looks/searches beyond the matrix by excluding specific strings within the matrix headers.

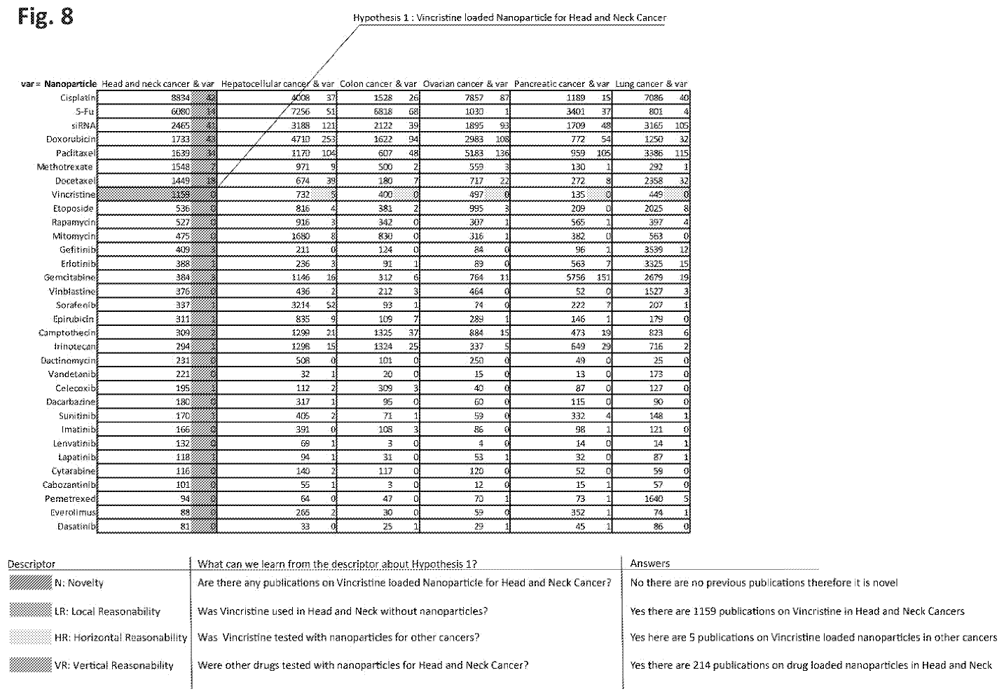

In the example shown in FIG. 8, hypothesis descriptors of novelty and reasonability in a merged comparison matrix are defined. Various generated hypotheses are sorted in the matrix. Their novelty and reasonability (local, horizontal and vertical) are determined. To demonstrate the scoring ranking, one hypothesis is used as an example: “vincristine loaded nanoparticles for head and neck cancer” (“Hypothesis 1”). It can be seen that there are 1159 publications of the drug vincristine with head and neck cancer, but there are no publications that include nanoparticles in head and neck cancer together with vincristine. Therefore, it can be concluded that hypothesis 1 is novel (no publications, NOP=0) and with the starting assumption that it is has reasonability. We can now look at vertical and horizontal cells in the matrix of the ‘var’ type and two additional things can be learned: 1) head and neck cancer has used nanoparticles with other drugs and 2) vincristine was used in nanoparticles for other cancers. This can be quantified and it can be seen that there are five publications in the horizontal reasonability descriptors and 214 publications on the vertical reasonability descriptors together with 1159 papers in the local reasonability this scores as high in reasonability. The vertical and horizontal reasonability teaches about the feasibility, as it can be learned that it is feasible to make vincristine nanoparticles as well as use nanoparticles in head and neck cancer. Unpublished and published hypotheses can therefore be ranked without the need to review any publication. Thus, in this example, it can be suggested that vincristine loaded nanoparticles for head and neck cancer is a reasonable and novel hypothesis and when tested should be successful.

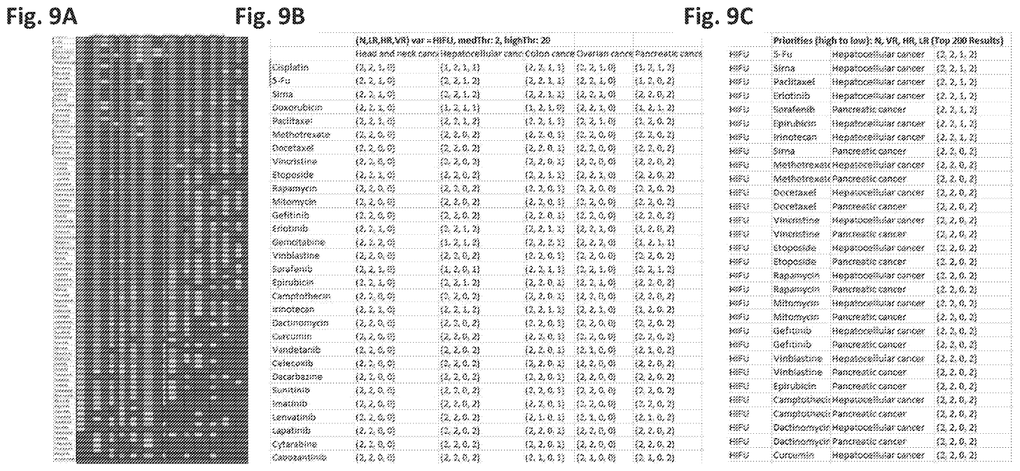

In the example shown in FIGS. 9A-C, the score of novelty and reasonability is evaluated automatically on a whole matrix. In FIG. 9A, the first step is to create a merged comparison matrix using the determined search terms. Next, the second step (FIG. 9B) is to calculate for each cell in the matrix using the thresholds determined by the user (in this example, high threshold=20, medium threshold=2), similarly to shading/colorization of the matrix (high and medium thresholds). In the third step (FIG. 9C), the hypotheses (cells) are ranked by user-defined priorities. In this example, the ranking priority was by N followed by VR, HR and finally LR, to identify most novel, most reasonable and most feasible hypotheses.

It is shown in FIGS. 9A-C, that novelty and reasonability can be evaluated using a score from 0 to 2 whereby 0 is low, 1 is medium, and 2 is high. FIG. 9A show the initial comparison matrix of cancers and drugs, and the additional search term (var) is “high intensity focused ultrasound” or HIFU. Using the same method described above, using local, vertical and horizontal reasonability as well as novelty, the algorithm scans the whole matrix and present the N, LR, HR, and VR score of each cell in the matrix (FIG. 9B). The hypotheses are then sorted by the desired parameters. In this example they are ranked by novelty first and then local reasonability. In this manner tens of thousands of hypotheses can be scanned and ranked by the novelty and reasonability descriptors. In FIG. 9C it is shown, for example, that HIFU combined with paclitaxel in hepatocellular cancer is highly reasonable and should work even though it was never published before.

### Example 7—Finding Novel and Reasonable Hypotheses with Three Variables Comparison Matrices

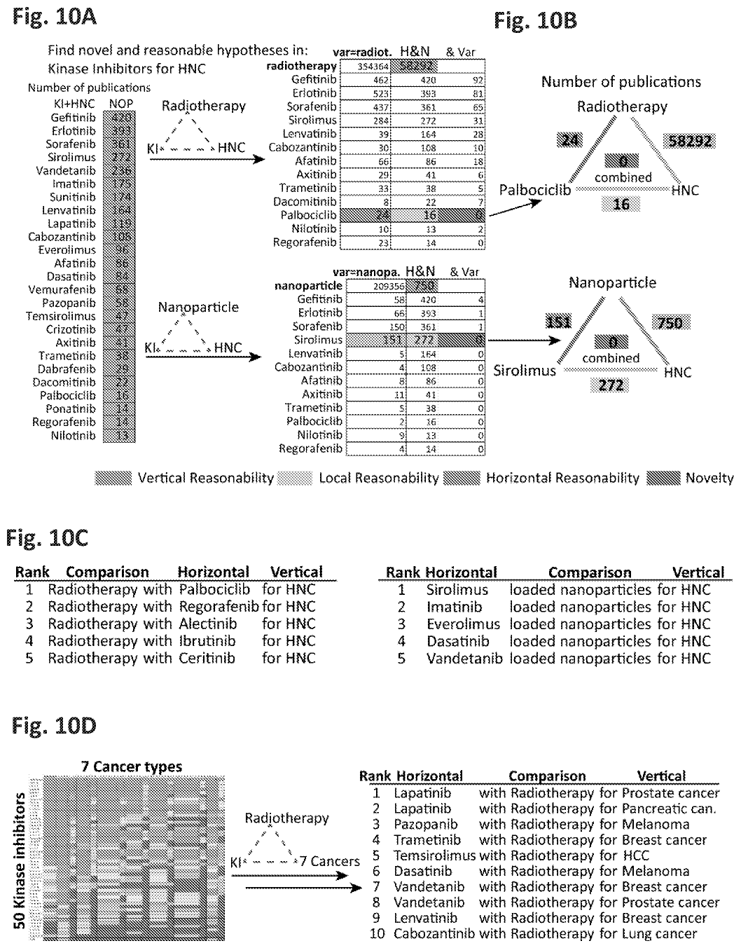

Another way of finding novel and reasonable hypotheses in biomedicine is to take a true and known hypothesis and add a novel element to it. In other words, to take something known and build an additional layer of complexity and novelty on it. In this way, starting with a hypothesis of two components can generate a three-component hypothesis. The analysis of the publications between the three components can provide insights on the reasonability and feasibility of the novel hypothesis, a scoring method is termed herein ‘triangulation’. As an example, all possible KIs in Head and Neck Cancer (HNC) were looked at and sorted by the highest NOP (Mg. 10A). Then, a novelty element was added to search, whereby the additional constant string “Radiotherapy” was added to the search list of KIs in HNC. This generates the comparison matrix, which juxtaposes the NOP of all possible pair combinations in the trio, KIs-HNC-Radiotherapy (FIG. 10A, right hand panel). It was hypothesized that if every pair has high NOP then the trio is reasonable even if it is an unpublished hypothesis. In this example, it is shown that the trio HNC-Palbociclib-Radiotherapy has no publications even though every possible pair of the trio has multiple publications (>15) (FIG. 10B). Within a trio, as detailed in above, there are three possible pairs (“descriptors”) that can be used to score the reasonability and novelty: local reasonability (LR) in this example, KI-HNC, vertical reasonability (VR), in this example, Radiotherapy-HNC, and horizontal reasonability (HR), KI-Radiotherapy. As detailed above, scoring the novelty and reasonability, allows the ranking of hypotheses by their descriptor scores. The scores range from “0” (low) to “2” (high), with “1” as medium, and sensitivity thresholds are defined by the user. The user can decide how many papers indicate novelty/reasonability. In this example, the most novel and reasonable hypothesis was HNC-Palbociclib-Radiotherapy which was validated with in a standard literature search. This validation process revealed a growing interest in palbociclib with radiation in many cancers, including a phase I/II dose escalation study of palbociclib in combination with cetuximab and radiation therapy for locally advanced squamous cell carcinoma of the Head and Neck (ClinicalTrials.gov Identifier: NCT03024489). Thus, by generating a comparison matrix and then analyzing the number of publications between its pair-elements, it is possible to identify and rank reasonable and feasible hypotheses even if they are unpublished. The same process with was repeated using the search string “nanoparticle” instead of “radiotherapy”, in order to find hypotheses where the KIs are encapsulated in a nanoparticle for HNC. Again, hypotheses that are novel and reasonable were found (FIG. 10B). All the hypotheses including KIs in HNC with ‘radiotherapy’ or ‘nanoparticle’ were ranked. The top five hypotheses ranked by their novelty and reasonability scores are presented in FIG. 10C. An evaluation of these ten hypotheses was performed with a standard literature review. In addition, biomedical researchers were asked to score these hypotheses in the same scale of ALMA (while blinded to results obtained by ALMA). ALMA ranking was compared to the ranking of researchers and seven out of the ten hypotheses (70%) were identically ranked and all of the other three hypotheses were ranked lower by humans even though supporting references could be found for all generated hypotheses. The search was then expanded/extended to 50 KIs in 7 additional cancers, and the top ten novel and reasonable KI-Cancer-Radiotherapy hypotheses are presented in FIG. 10D, based on the extended reasonabilities.

### Example 8. ALMA Guided Experiments Using Available Inventory Drugs and Cells

### Materials and Methods:

### Preparation of Indocyanine Nanoparticles

1.05 ml of each drug, dissolved in DMSO (10 mg/ml), was added drop-wise to a 0.6 ml aqueous solution containing IR783 (Sigma Aldrich, 2 mg/ml) and 0.1 mM sodium bicarbonate. The solution was centrifuged (20,000 G, 30 min), and the pellet was re-suspended in 1 ml of de-ionized water. In cases of a pellet that was difficult to re-suspend, it was bath sonicated for 3-5 minutes. Dynamic light scattering (DLS) and zeta potential measurements were conducted using a Zetasizer Nano ZS (Malvern).

### Cell Culture

Human osteosarcoma MG-63, U2OS cell lines were kind gift from David Meiri, and head and neck FaDu cell line were a kind gift of Moshe Elkabetz. These cells were incubated under standard conditions of 37° C., 5% CO2, and 95% humidity. MG-63 and U2OS cells were cultured in RPMI-1640 (Biological Industries) containing 10% fetal bovine serum, 2 mM L-Glutamine (Biological Industries) and 1% penicillin/streptomycin (Biological Industries).

FaDu cell line were cultured in DMEM (Biological Industries) containing 10% fetal bovine serum, 2 mM L-Glutamine (Biological Industries) and 1% penicillin/streptomycin (Biological Industries).

### Cell Viability Assay by MT

5000 cells per well in 0.2 ml growth media were seeded in a 96-well plate and allowed to attach for 24 hours. After 24 hours the cells were exposed to logarithmic gradient of drugs (Gemcitabine, Sorafenib, Nilotinib, Carfilzomib, Nintedanib, Trametinib, Cabozantinib, Ponatinib, Infigratinib, Duvelisib).

Cell survival for the cell lines was assayed after 3 days from adding the drugs. For the U20S and MG-63 by adding 50 W of MT solution (5 mg/ml) in DDW to each well. After 3 hours, the solution was removed and 200 μl of DMSO was added. For the Fadu cell line by adding 30 μl of MTT solution (5 mg/ml) in DDW to each well. After 1 hour, the solution was removed and 100 μl of DMSO was added to dissolve the formazan crystals. Cell viability was evaluated by measuring the absorbance of each well using a Synergy H1 (BioTek) plate reader at 570 nm relative to control wells.

### Fluorescence Microscopy

1000 cells per well in 0.2 ml growth media were seeded in a 96-well plate and allowed to attach for 24 hours. The cells were incubated for 2 hr with nanoparticle solution (50 μg/ml) and washed ×3 with PBS and then incubated again with HBSS buffer for imaging with BioTek LionHeart automated microscope in Cy7 channel to image IR783 dye in the particles.

In this example, it was sought to utilize ALMA to generate novel and reasonable hypotheses from materials existing the lab. More specifically, ALMA was used to identify what has not been done (according to the literature) with the cell lines and drugs in the lab while focusing on the field of nanomedicine for drug delivery (FIG. 11A). A search matrix was generated with 50 drugs present in the lab and 15 cell lines (FIG. 11B). The search was focused on specific cancers and two search matrices were generated using the strings ‘osteosarcoma’ and ‘head and neck squamous cell carcinoma’ (HNSCC) and selected cell lines with more than 20 publications. Fadu was chosen for HNSCC and MG63 for osteosarcoma. A comparison matrix was generated with the word ‘nanoparticle’ to visualize what has and not been done with these cells and drugs in the context of nanomedicine. More than 50% of the drugs from the tested inventory have not been published with the MG63 and Fadu cell lines. The comparison matrix using the string ‘nanoparticle’ showed that only one drug (paclitaxel) from the inventory was published with all the cell lines (FIG. 11B, right panel). With the aim to conduct in vitro cell viability experiments, drugs that have five or fewer publications were selected with MG63 and Fadu cell lines. A focused in vitro screen of 10 of the drugs with a cell viability assay (MTT) was conducted and the cell viability results to the NOP were compared (FIG. 11C). The in-vitro screen demonstrated three highly potent drugs for MG63, for which no information was identified in the literature. The most potent compound, carfilzomib (a drug approved for multiple myeloma), showed more than 95% cytotoxicity at low nanomolar concentrations and was only mentioned once with osteosarcoma and never with MG63 (FIG. 11C, top). Potent growth inhibition was also observed for the MEK inhibitor, trametinib, with only two publications with osteosarcoma and no publication for MG63. In Fadu cells, carfilzomib was also the most potent molecule in the in-vitro screen, although it seemed less potent than in MG63 with only 64% cytotoxicity at nanomolar concentration (FIG. 11C, bottom). In order to prepare nanoparticles from the most potent unpublished drug, carfdzomib, a previously published method of high loading nanoparticle prediction algorithm from molecular structure was used. According to this algorithm, carfdzomib was predicted to form <150 nm indocyanine stabilized nanoparticles with high drug loading. Indeed, the published protocol for nanoparticle preparation was used to successfully prepare both carfilzomib and sorafenib (as published control) nanoparticles with more than 80% loading efficiency. The size and charge characterization of the nanoparticles was 120 nm and −30 mV, respectively (FIGS. 11D-E). The in vitro cytotoxicity of the nanoparticles was tested and compared to the free drug (FIG. 11F). The results indicated that MG63 are extremely sensitive to carfilzomib and its indocyanine nanoparticle formulation (Car-INP), and it was highly active even in extremely low concentrations of down to 1×10-25 mg/ml (FIG. 11G). Fadu cells were less sensitive but the nanoparticle formulation had a marked advantage over the free drug at low concentrations (FIG. 11F). The uptake of the Car-INP particles was then tested in vitro (FIG. 11H) and marked nanoparticle uptake was observed after 2 h of incubation for both cells, which according to the previous studies might be explained by their high CAV1 expression.

### Example 9: ALMA Guided Search for New Research Projects in Biomedicine

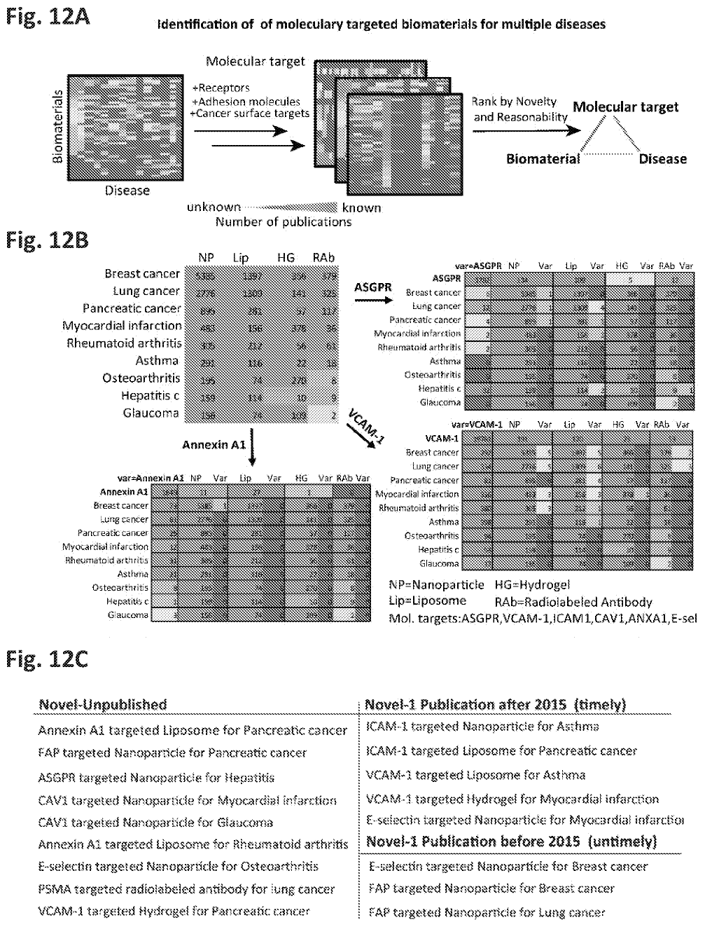

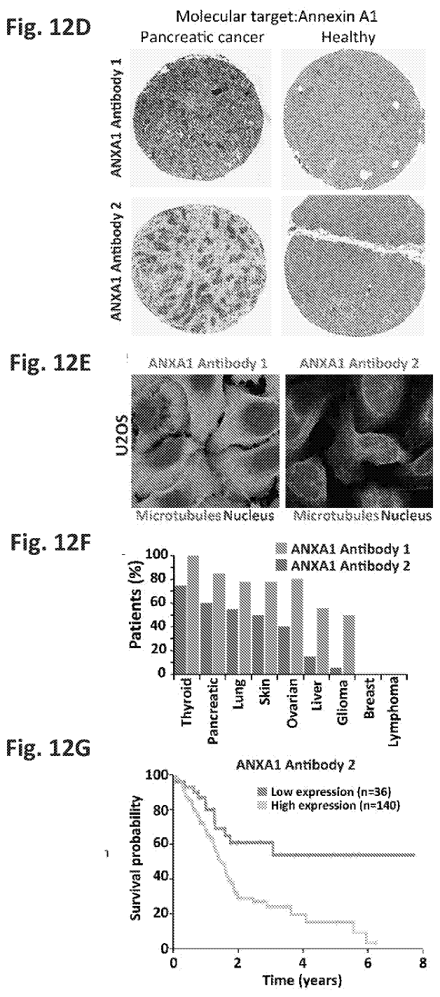

In this example, ALMA was used to automatically generate new biomedical research projects with additional complexity. The focus was on the use of molecularly targeted biomaterials for treatment or diagnosis of various diseases (FIG. 12A). This is a common type of biomedical research question with a combinatorial structure, for example, ‘Biomaterial A modified with targeting ligand B in disease C’, where each variable can be replaced by words from categorized lists of biomaterials, ligands and, diseases. The most common use is for a biomaterial to bind a molecular target in a certain disease to deliver drugs or diagnostic agents. As a demonstration, only four types of materials which are known for their use as vehicles for molecular targeting were selected, namely: hydrogels, liposomes, nanoparticles, and radiolabeled antibodies. Nine different diseases were selected: three cancers (breast, pancreatic and lung), two autoimmune diseases (osteoarthritis and rheumatic arthritis), myocardial infarction, asthma, hepatitis c and, glaucoma. Five distinct surface proteins that are potential targets in inflammation and cancer from different classes were selected, including endothelial adhesion molecules (E-selectin, VCAM1 and, ICAM1), a lipid binding protein (Annexin A1), caveolae scaffold protein (CAV1), a fibroblast activation enzyme (FAP) and a galactose receptor (ASGPR). To find novel and reasonable hypotheses in this space, a regular search matrix was first generated (9 diseases with 4 types of biomaterials) which contains all the possible diseases-biomaterials combinations (FIG. 12B). This matrix shows that almost all combinations have some publications. The highest NOPs in this matrix are for nanoparticles for all three cancers, which indicates that cancer nanomedicine is the center of knowledge as the most studied field in this space. The least explored space with lowest NOPs was for radiolabeled antibodies for glaucoma, hepatitis and osteoarthritis. This matrix was used as a basis for multiple comparison matrices with the list of molecular targets. This creates a three element hypotheses combination and the basis of the scoring system by triangulation (Mg. 12B). It is clear that the addition of the targets dramatically reduced NOP for most hypotheses to zero (red). In most leading hypotheses, such as nanoparticles for breast cancer, the resulting NOP represents only a small fraction of the studies containing just two elements (without targeting). The scoring matrix was used to rank the hypotheses according to the following sensitivity thresholds: novelty score (51 publication) and reasonability score (≥10 publications in every pair combination) (FIG. 12C). The top 20 novel and reasonable hypotheses were explored and identified which of them have no publications at all and which of them have just one publication, and when was it published. It was speculated that if a hypothesis has one publication in the past 5 years it is relatively novel and timely but if it was published more than 5 years ago it might indicate that it did not develop into fruitful research. In order to evaluate the reasonability and novelty of these generated hypotheses, they were proposed as research proposals. As selected portion of such proposed research proposal were defined by researchers as reasonable enough to investigate.

Presented below is an example of one such novel hypothesis “Annexin A1 targeted liposomes for pancreatic cancer” which was evaluated for its reasonability. For validation of the target, Annexin A1 (coded by ANXA1) in pancreatic cancer, the human protein atlas database (HPA) (http://www.proteinatlas.org) was used. In this database, there are multiple staining of hundreds of proteins with different antibodies for each target. Differential staining of ANXA1 in healthy pancreas compared to pancreatic cancer patients using two antibodies (FIG. 12D) was found. One antibody seems to stain the membrane stronger than the other, but both showed high staining in cancer patients as compared with healthy controls. The difference between the two antibodies was seen clearly in cellular expression of ANXA1 in vitro (U2OS osteosarcoma cells) where Antibody 1 (HPA011271) showed high membrane staining and Antibody 2 (CAB013023) had positive weak intracellular staining (FIG. 12E). HPA was also investigated for the expression of ANXA1 in nine different cancers type with the two antibodies and for both, pancreatic cancer was ranked as one of the top cancers expressing ANXA1 (FIG. 12F). Furthermore, it was also found that high expression of ANXA1 is correlated with poor survival with a 5-year survival probability of 18% and 56% for high and low expression respectively (FIG. 12G, P=0.0025). A comprehensive literature survey was then performed, and several evidences were found in the literature of ANXA1 involvement in pancreatic cancer progression. In addition, ANXA1 was studied as a target for drug delivery in several tumors such as colon, lung, prostate and, breast cancer, but never in pancreatic cancer. In addition, it was reported to be involved in a transvascular pumping mechanism, which allows rapid uptake into dense tumors. In these studies, ANXA1 was targeted with antibodies or with a short peptide named IF7 that was conjugated to polymers and nanoparticles. Interestingly, most of the papers studying ANXA1 with liposomes did not use them as vehicles for targeting but used them as research tools, as ANXA1 is a known lipid binding protein. It can be therefore reasonable to suggest that the combination of liposomes and targeting peptide or an antibody could have a higher affinity to Annexin A1 than with nanoparticles or polymers, possibly achieving better tumor targeting.

### Example 10: Temporal Analysis of Hypotheses

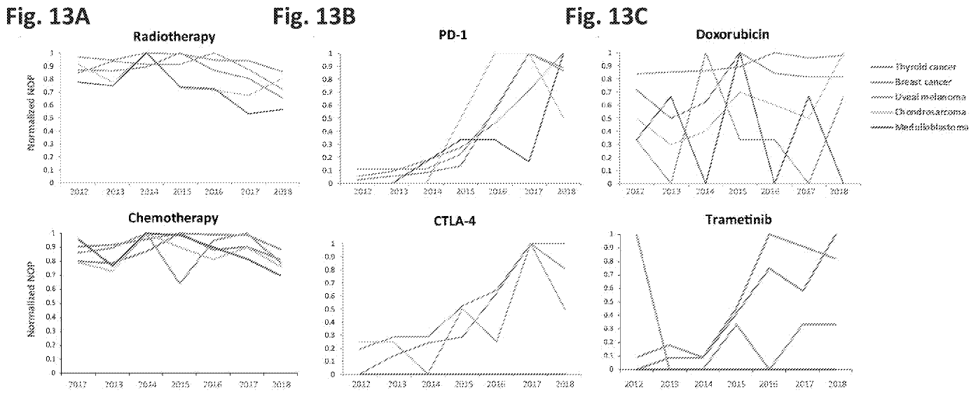

An important factor for literature review and scientific research in general, is to know which hypothesis is emerging as an important truth or is trending in a scientific field. It could also be regarded as another aspect of novelty. To this end, the ALMA's automated search may further be used to extract the number of publications per year (temporal distribution). As shown in FIGS. 13A-C, the yearly publications of five different cancers together with six different variables (concepts) are presented. The number of publications (NOP) was normalized to the highest NOP of the specific cancer. In FIG. 13A, variables of traditional pillars of cancer treatments (chemotherapy and radiotherapy) are presented. These are relatively constant and in slight decline. In contrast, as can be seen in FIG. 13B, emerging concept of novel treatments are based on immunotherapy using the targets: PD-1 and CTLA-4. In FIG. 13C, an example of mixed trends that are specific for the tumor types can be seen.

Thus, the ALMA algorithm can be used to identify trends and temporal changes of various hypotheses.

### Example 11—Temporal and Geographical Analysis of Biomedical Hypotheses

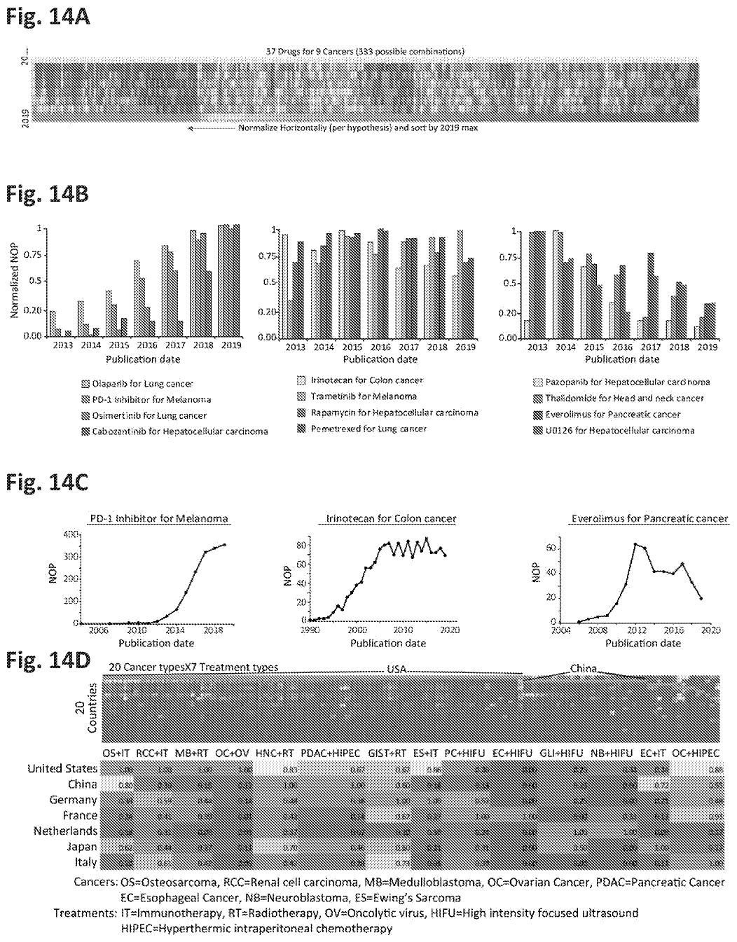

In this example, it was sought to demonstrate the ability to analyze the temporal and/or geographical trend of biomedical hypotheses. To this aim, the hypotheses text generator was used to generate all possible combinations between 37 drugs and 9 cancer types (333 combinations). Then, a general search matrix of the 333 hypotheses was created, sorted by NOP and selected only published hypotheses (NOP≥1) to generate another search matrix together with the year of publication from 2013 until 2019. The matrix was normalized horizontally in order to visualize which year had the maximal amount of publications per hypothesis, as shown in FIG. 14A. Then it was sorted to identify the hypotheses, which only in 2019 had the highest amount of publications. The NOP was plotted over time for hypotheses peaking in 2019, stable in the past 6 years and declining (FIG. 14B). In the trending hypotheses, many combinations of PD-1 inhibitors were found, which is a well-known growing field of research. The third generation, irreversible EGFR inhibitor Osimertinib was also identified, which is doubling its number of publications every year for the past three years. From a short literature review, it seems that osimertinib is more effective than chemotherapy combination of pemetrexed and cisplatin. Cabozantinib is also trending in several cancers and significantly in hepatocellular carcinoma. It had showed clinical benefit in patients that developed resistance to sorafenib as first line therapy. Olaparib in lung cancer had steadily doubled its publications in the past four years. It is mainly an established drug for ovarian and breast cancer (stable hypothesis) and in small cell lung cancer, it is being investigated as a combination companion drug and was tested with both chemotherapy, radiotherapy, and targeted therapy. Several declining hypotheses were found, such as pazopanib in HCC and everolimus for pancreatic cancer. PubMed's results-per-year feature was used to show representative hypotheses from their very beginning. The results are presented in FIG. 14C.

In addition to temporal analysis, it is also possible to interrogate the geographic distribution of biomedical hypotheses in a similar manner. Therefore, instead of generating a search matrix of hypotheses vs years, a search matrix of ‘hypotheses vs countries’ was generated (“geographical matrix”). The text generator was used to first generate all possible hypotheses involving 7 unconventional treatment types in 20 different cancer types (140 possible combinations), and only published hypotheses (NOP≥1) were selected for further geographic analysis. A new search matrix was generated using the list of published hypotheses together with a list of the 20 countries and the matrix was normalized per hypothesis (horizontal normalization) to identify in which country this hypothesis is most popular (FIG. 14D). The majority of hypotheses had their highest NOP in the united stated with 90 of 140 hypotheses (64.3%) and China with 26 of 140 (18%). A focused representation of the original matrix was generated to show which hypotheses are unique to which country. For example, it is shown that studies of hyper-thermic intraperitoneal chemotherapy (HIPEC) for ovarian cancer are mostly popular in Italy and France while the use of an oncolytic virus for the same cancer is almost exclusive to the US. High intensity focused ultrasound (HIFU) for glioma is unique to the Netherlands and the use of immunotherapy in esophageal cancer is unique to Japan. A unique hypothesis for Germany is using radiotherapy in gastrointestinal stromal tumors (GIST).

Thus, as demonstrated herein, the use of ALMA to generate data on the geographical and temporal distribution of biomedical hypotheses can be a valuable tool for decision making regarding choice of research project topics and suggest ways to form collaborations.

### Example 12: Evaluating and Ranking Drug Candidate for COVID-19 by Novelty and Reasonability Score

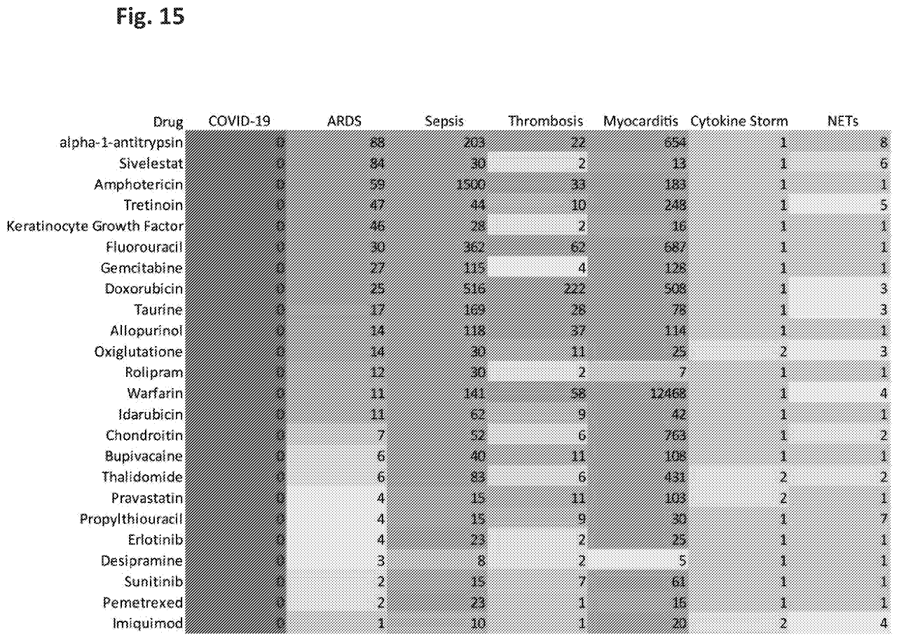

In this example, the hypothesis text generator was used to generate search matrices of drugs with several COVID-19 Related Keywords (CRK), including RNA viruses, antiviral therapy, cytokine storm, neutrophil extracellular traps, acute respiratory distress syndrome, sepsis, myocarditis, coagulation. Top COVID-19 co-occurring drugs were pulled together, and all the matrices were sorted by their occurrence with CRK and COVID-19. In this manner, the already published/known drugs for COVID-19 were separated from the unpublished drugs. The unknown COVID-19 drugs were ranked by their reasonability score which was calculated by the CRK cumulative occurrence (FIG. 15).

Apart from the current treatments with antivirals/anti malaria drugs, the most reasonable drugs in the list were MTOR inhibitors sirolimus/rapamycin and everolimus, immunosuppressant cyclosporin, anti proteases and antibiotics, steroid prednisolone and kinase inhibitor baricitinib. Within the top 10 COVID-19 reasonable drugs, two were never published with COVID-19 (cyclosporine, prednisolone).

### Example 13: Determining a High Resolution Combination Therapy (HRCT) Using ALMA

In this example, the HRCT generation workflow included such questions as: what is the top drug for KRAS driven Lung Cancer (answer: Trametinib); What drug goes with Trametinib? (answer: Dabrafinib). What treatment goes with trametinib? Answer: Immunotherapy; What goes with immunotherapy? Answer: Radiotherapy, and so on. The results provided by ALMA are used to generate the detailed treatment regime which is presented in FIG. 18. The treatment regime is personalized to a specific patient having a specific type of caner (lung cancer, stage 2), with specific genetic mutations at KRAS and PTEN. The treatment regime illustrated in FIG. 18, lists the various drug treatments (including various drugs administration); treatment procedures (including, radiotherapy, immunotherapy, surgical procedures, psychotherapy), intervention procedures (such as specific diet, physical activity, etc.), as well as the sequence of the treatments and the temporal order of the treatments.

1. A computer implemented method for generating and ranking of hypotheses, based on a set of search terms, the method comprising:
obtaining two or more sets of search terms;
generating a plurality of combinations of search terms from the sets, each combination corresponding to a hypothesis;
for each of the plurality of combinations of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of at least one of a degree of novelty, a degree of feasibility, and a degree of reasonability of the hypotheses.

2. The method of claim 1, further comprising a step of performing an additional search using a second set of search terms or search variables on the sorted NOP matrix of the one or more selected generated hypotheses, to thereby generate a comparison matrix between the sorted NOP matrix and the results of the additional search.

3. The method of claim 1, further comprising presenting one or more of the matrix of the NOP, the sorted matrix of the NOP, and the ranking of the selected generated hypotheses.

4. The method of claim 1, wherein the hypothesis is a scientific hypothesis.

5. The method of claim 1, wherein each search term is at least one of a word, list of words, a sentence, a generic term, and a question.

6. The method of claim 1, wherein the selected combination of the search is structured as at least one of “one vs. many” and “many vs. many.”

7. The method of claim 1, wherein the search is performed using a web crawler, a web scraper, or an automated search tool.

8. The method of claim 1, wherein the electronic database is one of PubMed, Google Scholar, clinicaltrials.gov, Embase, and Semantic Scholars.

9. The method of claim 1, wherein the NOP matrix is visualized using a visual coding having adjustable threshold, based on the visualization parameters.

10. The method of claim 1, wherein the degree of reasonability comprises at least one of local reasonability (LR), horizontal reasonability (HR), and vertical reasonability (VR).

11. The method of claim 10, wherein the degree of reasonability further comprises at least one of extended horizontal reasonability (THR) and extended vertical reasonability (TVR).

12. The method of claim 10, wherein at least one of the degree of feasibility and the degree of reasonability are determined based on an adjustable threshold of number of publications.

13. The method of claim 12, wherein the adjustable threshold is user defined.

14. The method of claim 1, further comprising providing a numerical score based on the ranking of the hypothesis.

15. The method of claim 1, for identifying the temporal occurrence of hypotheses.

16. The method of claim 1, further comprising identifying the geographical distribution of hypotheses.

17. A computer implemented method for generation and ranking of hypotheses, based on a set of search terms, the method comprising:
obtaining a set of two or more search terms;
generating multiple hypotheses, based on a selected combination of the search terms;
performing a search for the generated hypotheses on one or more databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters; and
ranking the selected generated hypotheses based on the NOP matrix, wherein the ranking is indicative of at least one of the degree of novelty, a degree of feasibility, and a degree of reasonability of the selected generated hypothesis.

18. (canceled)

19. The method of claim 17 further comprising a user interface unit, a display unit and a communication unit.

20. (canceled)

21. A computer implemented method for determining a personalized high resolution treatment regime of a patient afflicted with a disease, the method comprising:
obtaining a set of two or more search terms related to the disease of the patient;
generating multiple hypotheses related to treatment of the disease, based on a selected combination of the search terms;
performing a search for the generated hypotheses on one or more suitable databases stored on a server, to determine the number of publications (NOP) for each generated hypothesis;
generating a matrix of the NOP of one or more selected generated hypotheses;
sorting the NOP matrix of the one or more selected generated hypotheses, based on one or more sorting parameters;
ranking the selected generated hypotheses based on the NOP matrix, wherein the ranking is indicative of at least one of a degree of novelty, a degree of feasibility, and a degree of reasonability of the selected generated hypothesis, to determine a first treatment;
repeating the search for one or more times with search terms related to at least one of the disease and the first treatment, to determine an additional one or more treatments; and
determining, based on the identified treatments, a personalized treatment regime for said patient.

22. The method according to claim 19, wherein the treatment is a combination therapy.

23. The method according to claim 19, wherein the patient is a cancer patient.

24. The method according to claim 21, wherein at least one of the first treatment and the one or more additional treatments are selected from at least one of a drug, an immunotherapy, a surgical procedure, radiotherapy, chemotherapy, psychotherapy, and lifestyle therapy.

25. The method according to claim 22, wherein the immunotherapy is one of antibodies based therapy and engineered T-cells.

26. The method according to claim 19, wherein the treatment regime further includes a spatial distribution sequence of at least one of the first and additional treatment.

27. The method according to claim 19, wherein the treatment regime further includes a nanoparticle formulation of at least one of the first and additional pharmacological treatment.

28. A computer implemented method for determining a personalized high resolution treatment regime of a patient afflicted with a disease, the method comprising:
obtaining two or more sets of search terms;
generating a plurality of combinations of search terms from the sets, each combination corresponding to a hypothesis related to treatment of the disease;
for each combination of search terms, searching on one or more electronic databases for the combination, thereby obtaining a number of publications (NOP) corresponding to the respective hypothesis;
generating a matrix with components indexed according to the hypotheses, each component assigned a value equal to the NOP of the combination of search terms corresponding to the respective hypothesis;
sorting the matrix according to one or more sorting criteria; and
ranking at least some of the hypotheses based on the sorted matrix, wherein the ranking is indicative of at least one of a degree of novelty, a degree of feasibility, and a degree of reasonability of the hypotheses, to determine a first treatment;
repeating the search for one or more times with search terms related to at least one of the disease and the first treatment, to determine an additional one or more treatments; and
determining, based on the identified treatments, a personalized treatment regime for said patient.

29. The method according to claim 26, wherein the treatment is a combination therapy.

30. The method according to claim 26, wherein the patient is a cancer patient.

31. The method according to claim 28, wherein at least one of the first treatment and the one or more additional treatments are selected from: a drug, an immunotherapy, a surgical procedure, radiotherapy, chemotherapy, psychotherapy, and lifestyle therapy.

32. The method according to claim 29, wherein the immunotherapy is one of antibodies based therapy and engineered T-cells.

33. The method according to claim 24, wherein the treatment regime further includes a spatial distribution sequence of at least one of the first and additional treatment.

34. The method according to claim 26, wherein the treatment regime further includes a nanoparticle formulation of at least one of the first and additional pharmacological treatment.

35. A system for automated generation of a hypothesis comprising a processor configured to:
obtain two or more sets of search terms;
generate a plurality of combinations of search terms from the sets, each combination corresponding to a hypothesis;
for each of the plurality of combinations of search terms, search on one or more electronic databases for the combination, thereby obtaining a number of publications (NOP) corresponding to the respective hypothesis;
generate a matrix with components indexed according to the hypotheses, each component assigned a value equal to the NOP of the combination of search terms corresponding to the respective hypothesis;
sort the matrix according to one or more sorting criteria; and
rank at least some of the hypotheses based on the sorted matrix, wherein the ranking is indicative of at least one of a degree of novelty, a degree of feasibility, and a degree of reasonability of the hypotheses.

36. The system of claim 33, wherein the processor is further configured to perform an additional search using a second set of search terms or search variables on the sorted NOP matrix of the one or more selected generated hypotheses, to thereby generate a comparison matrix between the sorted NOP matrix and the results of the additional search.

37. The system of claim 33, wherein the processor is further configured to present one or more of the matrix of the NOP, the sorted matrix of the NOP, and the ranking of the selected generated hypotheses.

38. The system of claim 33, wherein the hypothesis is a scientific hypothesis.

39. The system of claim 33, wherein each search term is at least one of a word, list of words, a sentence, a generic term, and a question.

40. The system of claim 33, wherein the selected combination of the search is structured as at least one of “one vs. many” and “many vs. many.”

41. The system of claim 33, wherein the search is performed using a web crawler, a web scraper, or an automated search tool.

42. The system of claim 33, wherein the electronic database is one of PubMed, Google Scholar, clinicaltrials.gov, Embase, and Semantic Scholars.

43. The system of claim 33, wherein the NOP matrix is visualized using a visual coding having adjustable threshold, based on the visualization parameters.

44. The system of claim 33, wherein the degree of reasonability comprises at least one of local reasonability (LR), horizontal reasonability (HR), and vertical reasonability (VR).

45. The method of claim 42, wherein the degree of reasonability further comprises at least one of extended horizontal reasonability (THR) and extended vertical reasonability (TVR).

46. The system of claim 42, wherein at least one of the degree of feasibility and the degree of reasonability are determined based on an adjustable threshold of number of publications.

47. The system of claim 44, wherein the adjustable threshold is user defined.

48. The system of claim 33, wherein the processor is further configured to provide a numerical score based on the ranking of the hypothesis.

49. The system of claim 33, wherein the processor is further configured to identify the temporal occurrence of hypotheses.

50. The system of claim 33, wherein the processor is further configured to identify the geographical distribution of hypotheses.

51. A non-transitory computer readable medium having stored thereon software instructions that, when executed by a processor, cause the processor to:
obtain two or more sets of search terms;
generate a plurality of combinations of search terms from the sets, each combination corresponding to a hypothesis;
for each of the plurality of combinations of search terms, search on one or more electronic databases for the combination, thereby obtaining a number of publications (NOP) corresponding to the respective hypothesis;
generate a matrix with components indexed according to the hypotheses, each component assigned a value equal to the NOP of the combination of search terms corresponding to the respective hypothesis;
sort the matrix according to one or more sorting criteria; and
rank at least some of the hypotheses based on the sorted matrix, wherein the ranking is indicative of at least one of a degree of novelty, a degree of feasibility, and a degree of reasonability of the hypotheses.

52. The non-transitory computer readable medium of claim 49, wherein the processor is further caused to perform an additional search using a second set of search terms or search variables on the sorted NOP matrix of the one or more selected generated hypotheses, to thereby generate a comparison matrix between the sorted NOP matrix and the results of the additional search.

53. The non-transitory computer readable medium of claim 49, wherein the processor is further caused to present one or more of the matrix of the NOP, the sorted matrix of the NOP, and the ranking of the selected generated hypotheses.

54. The non-transitory computer readable medium of claim 49, wherein the hypothesis is a scientific hypothesis.

55. The non-transitory computer readable medium of claim 49, wherein each search term is at least one of a word, list of words, a sentence, a generic term, and a question.

56. The non-transitory computer readable medium of claim 49, wherein the selected combination of the search is structured as at least one of “one vs. many” and “many vs. many.”

57. The non-transitory computer readable medium of claim 49, wherein the search is performed using a web crawler, a web scraper, or an automated search tool.

58. The non-transitory computer readable medium of claim 49, wherein the electronic database is one of PubMed, Google Scholar, clinicaltrials.gov, Embase, and Semantic Scholars.

59. The non-transitory computer readable medium of claim 49, wherein the NOP matrix is visualized using a visual coding having adjustable threshold, based on the visualization parameters.

60. The non-transitory computer readable medium of claim 49, wherein the degree of reasonability comprises at least one of local reasonability (LR), horizontal reasonability (HR), and vertical reasonability (VR).

61. The non-transitory computer readable medium of claim 58, wherein the degree of reasonability further comprises at least one of extended horizontal reasonability (THR) and extended vertical reasonability (TVR).

62. The non-transitory computer readable medium of claim 58, wherein at least one of the degree of feasibility and the degree of reasonability are determined based on an adjustable threshold of number of publications.

63. The non-transitory computer readable medium of claim 60, wherein the adjustable threshold is user defined.

64. The non-transitory computer readable medium of claim 49, wherein the processor is further caused to provide a numerical score based on the ranking of the hypothesis.

65. The non-transitory computer readable medium of claim 49, wherein the processor is further caused to identify the temporal occurrence of hypotheses.

66. The non-transitory computer readable medium of claim 49, wherein the processor is further caused to identify the geographical distribution of hypotheses.
